SEQUENCE LISTING (14 sequences)

SEQ ID NO: 1 - RNA, 55 nt, Artificial Sequence
  Synthetic oligonucleotide
ggaugcgguaccugacagcauccuaaacucaugguccauguuuguccauggacca

SEQ ID NO: 2 - RNA, 63 nt, Artificial Sequence
  Synthetic oligonucleotide
ggaugcgguaccugacagcaucuugaaauaaggacugaugcucaacaucagucugauaagcua

SEQ ID NO: 3 - RNA, 63 nt, Artificial Sequence
  Synthetic oligonucleotide
ggaugcgguaccugacagcaucuugaaauaaggacugaugcucaacaucagucugacacucca

SEQ ID NO: 4 - RNA, 64 nt, Artificial Sequence
  Synthetic oligonucleotide
ggaugcgguaccugacagcauccucccccgcauuccauggucaacccauggaauucaguucuca

SEQ ID NO: 5 - RNA, 23 nt, Artificial Sequence
  Synthetic oligonucleotide
ggaugcgguaccugacagcaucc

SEQ ID NO: 6 - RNA, 45 nt, Artificial Sequence
  Synthetic oligonucleotide
ggaugcgguaccugacagcauccucaacaucagucugcagccgag

SEQ ID NO: 7 - RNA, 45 nt, Artificial Sequence
  Synthetic oligonucleotide
ggaugcgguaccugacagcauccucaacaucagucugauaagcua

SEQ ID NO: 8 - RNA, 45 nt, Artificial Sequence
  Synthetic oligonucleotide
ggaugcugucagguaccgcauccucaacaucagucugcagccgag

SEQ ID NO: 9 - RNA, 45 nt, Artificial Sequence
  Synthetic oligonucleotide
ggaugcugucagguaccgcauccucaacaucagucugauaagcua

SEQ ID NO: 10 - RNA, 23 nt, Artificial Sequence
  Synthetic oligonucleotide
  misc_feature (1)..(1): 5′ triphosphate
ggaugcugucagguaccgcaucc

SEQ ID NO: 11 - RNA, 22 nt, Artificial Sequence
  Synthetic oligonucleotide
ucaacaucagucugauaagcua

SEQ ID NO: 12 - RNA, 21 nt, Artificial Sequence
  Synthetic oligonucleotide
acagcccaucgacugguguug

SEQ ID NO: 13 - RNA, 22 nt, Artificial Sequence
  Synthetic oligonucleotide
aacccauggaauucaguucuca

SEQ ID NO: 14 - RNA, 22 nt, Artificial Sequence
  Synthetic oligonucleotide
cugaagaacugaauuucagagg

(19) United States
(12) Patent Application Publication
(10) Pub. No.: US 2022/0168331 A1
(43) Pub. Date: Jun. 2, 2022

(54) PRR-Activating and MicroRNA-Inhibiting Molecules and Methods of Using Same

(71) Applicant: Duke University, Durham, NC (US)

(72) Inventors: Jaewoo Lee, Durham, NC (US); Youngju Lee, Durham, NC (US)

(73) Assignee: Duke University, Durham, NC (US)

(21) Appl. No.: 17/430,991

(22) PCT Filed: Feb. 18, 2020
(86) PCT No.: PCT/US2020/018700

§ 371 Date: Aug. 13, 2021

Related U.S. Application Data
(60) Provisional application No. 62/806,946, filed on Feb. 18, 2019.
(60) Provisional application No. 62/827,396, filed on Apr. 1, 2019.

(51) Int. Cl. A61K 31/713; A61P 35/00; A61P 37/04; A61K 45/06

(52) U.S. Cl. CPC A61K 31/713; A61K 45/06; A61P 37/04; A61P 35/00

(57) ABSTRACT

The present disclosure provides nucleic acid molecules, compositions, and pharmaceutical compositions comprising a pattern recognition receptor (PRR) agonist and a microRNA antagonist and methods treating or preventing cancer in a subject in need thereof, the method comprising administering to the subject a therapeutically effective amount of the nucleic acid molecules, compositions, and pharmaceutical compositions.

### CROSS REFERENCE TO RELATED APPLICATIONS

This application claims priority to U.S. Provisional Patent Application Ser. No. 62/806,946, filed Feb. 18, 2019, and U.S. Provisional Patent Application Ser. No. 62/827,396, filed Apr. 1, 2019, the contents of each of which are hereby incorporated by reference in their entirety.

### BACKGROUND OF THE INVENTION

### Field of the Invention

The present disclosure provides nucleic acid molecules, compositions, and pharmaceutical compositions comprising a pattern recognition receptor (PRR) agonist and a microRNA antagonist and methods treating or preventing cancer in a subject in need thereof, the method comprising administering to the subject a therapeutically effective amount of the nucleic acid molecules, compositions, and pharmaceutical compositions.

### Description of the Related Art

It has been previously demonstrated that certain pattern recognition receptor (PRR) agonists, such as polyriboinosinic:polyribocytidylic acid (pIC)^{1,2} and 5′-triphosphate (5′ppp) RNAs,^{3,4} were able to induce both immunogenic cell death (ICD) in cancer cells and type I interferon (IFN) production by innate immune cells and cancer cells. ICD is characterized by the release of high levels of innate immune stimulators, such as damage-associated molecular patterns (DAMPs), adenosine triphosphate (ATP), and cell-surface calreticulin.⁵⁻⁹ DAMPs activate innate immune receptors, such as PRRs, on innate immune and inflammatory cells to produce immune stimulatory cytokines/chemokines,^{10,11} whereas ATP and calreticulin act as “find-me” and “eat-me” signals, respectively, to recruit antigen presenting cells, such as dendritic cells (DCs), promote uptake and clearance of dead/dying tumor cells, and present tumor antigens to T cells.^{12,13}

Type I IFNs have a wide range of anticancer activities, including augmentation of T cell- and natural killer (NK) cell-mediated cytotoxicity, upregulation of MHC-peptide complexes and T cell-recruiting chemokines (CXCL9 and CXCL10), and inhibition of cancer cell growth and angiogeneis.^{14,15} These DAMPs, find-me/eat-me signals, and type I IFNs cooperate to induce adaptive anticancer immune responses after treatment with ICD-inducing PRR agonists.¹⁶ Thus, these ICD-inducing PRR agonists can convert a nonimmunogenic tumor to an immunogenic tumor and synergize with immune checkpoint inhibitors to induce long-lasting anticancer immune responses. 5,7,10,17-19

PRRs are essential receptors of innate immune and inflammatory cells to recognize harmful insults, such as infection and tissue injury, and to trigger anti-infectious immunity and wound healing.^{20,21} A variety of PRRs are expressed in the cytoplasm, endo/lysosomal compartments, and on the cell surface. Each PRR binds to a distinct molecular pattern that is present on pathogens and damaged tissues, called pathogen-associated molecular patterns (PAMPs) and DAMPs, respectively.²² For example, retinoic acid-inducible gene I (RIG-I) and melanoma differentiation-associated protein 5 (MDA5) are cytoplasmic RNA helicases that recognize uncapped 5′ppp single-stranded RNAs (ssRNAs) and long double-stranded RNAs (dsRNAs), respectively.²³⁻²⁵ Stimulator of interferon genes (STING) is an endoplasmic reticulum-resident adaptor protein that binds to cyclic-di-nucleotides such as cyclic GMP-AMP (cGAMP).²⁶ Toll-like receptors (TLRs) are membrane-associated receptors that are located on the cell surface or in endo/lysosomal compartments.²⁷ TLRs 2, 4, 5, 6, and 11 bind to PAMPs (e.g., lipopolysaccharide (LPS), bacterial lipoproteins, peptidoglycan) and DAMPs (e.g., heparan sulfate, high mobility group box 1 protein (HMGB1)), whereas TLRs 3, 7, 8, and 9 recognize bacterial, viral, and cellular DNAs and RNAs.²⁸ The PRR downstream signaling culminates in the activation of various transcription factors, such as nuclear factor-κB (NF-κB), activator protein 1 (AP-1), and IFN regulatory factors (IRFs), resulting in the expression of cytokines/chemokines, type I IFNs, and IFN-stimulated genes (ISGs) and the facilitation of wound healing, virus/bacteria clearance and anticancer responses.²⁹⁻³²

To prevent unwanted onset of pathological inflammation, persistent stimulation of PRRs is highly regulated by the “PRR tolerance” mechanisms that lead to a hyporesponsive state of PRRs due to a desensitization of PRR signaling. PRR tolerance is induced by various self- and cross-regulatory mechanisms.³³ For example, mouse and human innate immune cells treated with LPS upregulate negative-feedback regulators of TLR signaling, such as IRAK-M,³⁴ A-20,³⁵ pellino-3,³⁶ SH2-containing inositol phosphatase (SHIP),³³ and immune regulatory microRNAs (miRs) (miR-21³⁷ and miR-146a³⁸), as early as 3-7 h after LPS treatment and remaining for 5-7 days. These negative-feedback regulators interfere with TLR4 downstream signaling, leading to the inhibition of NF-κB and IRFs and the suppression of proinflammatory cytokine and IFN gene expression. Interestingly, these negative-feedback regulators not only desensitize TLR4, but also other types of TLRs.³⁹

Alternatively, TLR activation and IFN treatment upregulate the expression of indoleamine 2,3-dioxygenase (IDO) that converts the essential amino acid tryptophan into the downstream catabolite kynurenine (Kyn), resulting in the activation of a transcription factor aryl hydrocarbon receptor (AHR) in various immune and nonimmune cells.⁴⁰⁻⁴² This IDO-Kyn-AHR pathway of tryptophan metabolism signaling regulates innate and adaptive immune responses by various mechanisms, including the induction of tolerogenic APCs,⁴³′⁴⁴ the inhibition of antigen-specific T cell and NK cell proliferation and activation,^{45,46} and the induction of Treg and Th17 cells.⁴⁷⁻⁴⁹ Moreover, the IDO-Kyn-AHR pathway inhibits the expression of proinflammatory cytokines (TNFα and IL-6) and type I IFNs and enhances the expression of anti-inflammatory cytokines (IL-10 and TGF-β) in innate immune cells, thereby inducing PRR tolerance.⁵⁰⁻⁵³

Unlike non-malignant cells, certain cancer cells constitutively express IDO and miR-21 that contribute to tumor progression, metastasis, resistance to anticancer therapy, and tumor immune escape.⁵⁴⁻⁵⁷ Such pre-existing and inducible PRR tolerance signaling and tryptophan metabolism signaling in cancer cells and innate immune cells diminish therapeutic effects of ICD-inducing PRR agonists. Accordingly, there exists a need in the art for novel molecules and methods to improve the therapeutic effects of ICD-inducing PRR agonists and to overcome tumor-associated PRR tolerance.

### SUMMARY OF THE INVENTION

In a first aspect, the present invention provides a nucleic acid molecule comprising a pattern recognition receptor (PRR) agonist and a microRNA antagonist. In certain embodiments of the first aspect of the invention, the PRR agonist is a RIG-I agonist. In certain embodiments, the micro RNA antagonist is a miR-21 antagonist.

In a second aspect, the present invention provides a composition comprising the nucleic acid molecule of the first aspect of the invention. In a third aspect, the present invention provides a pharmaceutical composition comprising the nucleic acid molecule of the first aspect of the invention and a pharmaceutically acceptable carrier and/or excipient.

In a fourth aspect, the present invention provides a composition comprising a PRR agonist/microRNA antagonist conjugate molecule. In certain embodiments of the fourth aspect of the invention, the PRR agonist is a protein, lipopolysaccharide, or small molecule.

In a fifth aspect, the present invention provides a method of treating or preventing cancer in a subject in need thereof, the method comprising administering to the subject a therapeutically effective amount of the nucleic acid molecule of the first aspect of the invention such that the cancer is treated or prevented in the subject.

In a sixth aspect, the present invention provides a method of treating a RIG-1-therapy resistant cancer in a subject, the method comprising, administering to the subject a therapeutically effective amount of the nucleic acid molecule of the first aspect of the invention such that the cancer is treated in the subject.

In a seventh aspect, the present invention provides a method of sensitizing a cancer cell to a PRR agonist, the method comprising exposing the cancer cell to the nucleic acid molecule of the first aspect of the invention.

In an eighth aspect, the present invention provides a method of improving an anticancer innate immune response to a cancer cell, the method comprising exposing the cancer cell to the nucleic acid molecule of the first aspect of the invention.

In a ninth aspect, the present invention provides a method of mitigating miR-21 or miR-146a interference with the antitumor effects of a RIG-I agonist in a cancer cell, the method comprising exposing the cancer cell to the nucleic acid molecule of the first aspect of the invention.

In a tenth aspect, the present invention provides a kit for the prevention and/or treatment of a cancer in subject, the kit comprising a composition according to the second or third aspect of the invention and instructions for use.

### BRIEF DESCRIPTION OF THE DRAWINGS

FIGS. 1A-1C. B16 melanoma cells are sensitive and resistant to RIG-I agonists. (FIG. 1A) cytotoxicity, (FIG. 1B) IFN-β production, and (FIG. 1C) tumor suppressor proteins and premiR-21 levels in B16-F0 and B16-F10 cells following ICR4 treatment and overnight incubation.

FIG. 2. Differential sensitivity of cancer and normal cells to RIG-I agonist ICR4: % Cytotoxicity.

FIGS. 3A-3D. Anti-tumor effects of RigantmiR. (FIG. 3A) Sequence of RIG-I agonist ICR4, RigantmiR-21, and seed mutant RigantmiR-21. (FIG. 3B) Secondary structure of RigantmiR-21 and seed mutant RigantmiR-21. RIG-I agonist ICR4 and anti-miR-21 were combined to form a RigantmiR-21. Line: ICR4. Dots: anti-miR-21 sequence. Squares: mutation of seed sequence. (FIG. 3C) Expression of miR-21 target genes and (FIG. 3D) IFN-β production following transfected once or twice with RigantmiR-21, mutant RigantmiR-21, anti-miR-21, or ICR4. (FIG. 3E) Survival rates following intratumoral injections of the indicated RNA complexes into B16-F10 melanoma-bearing immunocompetent mice.

FIGS. 4A-4C. RignatmiR-21 improves IFN-β production by mouse pancreatic cancer cells and sarcoma. IFN-β production in (FIG. 4A) KPC-4839 cells, (FIG. 4B) PANC-02 cells, and (FIG. 4C) mouse sarcoma cell lines.

FIGS. 5A-5B. Repeated treatments with RignatmiR-21 but not ICR4 gradually increase IFN-β production by mouse cancer cells. (FIG. 5A) Cytotoxicity levels three days after first transfection at various RNA concentrations. (FIG. 5B) IFN-β production one day after first and second transfection at 12.5 nM RNA.

FIGS. 6A-6B. RNA-sensing pattern recognition receptor agonists conjugated with anti-miR-21. (FIG. 6A) Sequences of RNA-sensing pattern recognition receptor agonists ICR2 and ICR2AS conjugated with anti-miR-21. (FIG. 6B) IFN-β production in B16-F10 mouse melanoma cells.

FIGS. 7A-7B. Repeated treatments did not improve innate immune stimulatory and anticancer effects of MDA5 agonists. (FIG. 7A) IFN-β production by the cells one day after each transfection. (FIG. 7B) Survival rate following multiple treatments with MDA5 agonists.

FIG. 8. Innate immune receptor activation or tryptophan signaling activation renders cancer cells resistant to MDA5 agonists: Relative IFN-β production.

FIGS. 9A-9B. Activation of cytoplasmic and surface innate immune receptors renders innate immune cells self- and cross-resistant to pattern recognition receptor agonists in a tryptophan metabolism signaling-dependent manner. (FIG. 9A) IFN-β production in mouse BM-DC cells. (FIG. 9B) Relative IFN-β production in mouse macrophages.

FIG. 10. Treatment with tryptophan metabolism signaling inhibitors improves innate immune stimulatory activities of MDA5, RIG-I, and STING agonists: IFN-β production.

FIG. 11. RignatmiR-21 and RigantmiR-146a differentially induce IFN-β production by different cancer cells: IFN-β production in 4T1 mouse breast cancer cells, B16-F10 mouse melanoma cells, 403688 mouse sarcoma cells, and WM266.4 human melanoma cells transfected with RIG-I agonists.

FIGS. 12A-12C. RignatmiR-21 synergizes with anti-PD-1 antibody for the treatment of sarcoma. (FIG. 12A) IFN-β production 24 h after treatment with RignatmiR-21 in murine sarcoma 402230 cell line. (FIG. 12B) Cytotoxicity 72h after treatment with RignatmiR-21 in murine sarcoma 402230 cell line. (FIG. 12C) Percent survival of 402230 sarcoma-bearing immunocompetent mice.

### DETAILED DESCRIPTION OF THE INVENTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

As used in the specification, articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result. The term “about” in association with a numerical value means that the numerical value can vary plus or minus by 5% or less of the numerical value.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other integer or step or group of integers or steps.

As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

Unless otherwise defined, all technical terms used herein have the same meaning as commonly understood by one of ordinary skill in the art to which this disclosure belongs.

### Modulating PRR Signaling

The inventors discovered that treatment with agonists for cytoplasmic RNA-sensing PRRs, such as retinoic acid-inducible gene I (RIG-I), induced immunogenic cell death (ICD) of various cancer cells, which was characterized by the production of Damage-Associated Molecular Patterns (DAMPs) and type I IFNs by cancer cells and the stimulation of dendritic cells. Interestingly, prior exposure of innate immune and cancer cells to DAMPs, PRR agonists, or tryptophan catabolite abrogated the ability of these cells to produce type I IFNs and inflammatory cytokines after treatments with nucleic acid-sensing PRR agonists. Co-treatment with indoleamine 2,3-dioxygenase (IDO) inhibitor and aryl hydrocarbon receptor (AHR) inhibitor is able to improve therapeutic effects of immunogenic cell death (ICD)-inducing PRR agonists.

Furthermore, the inventors rationally designed and generated a nuclease-resistant RNA molecule (named RigantmiR-21) that activates PRRs (e.g., RIG-I) and inhibit immune regulatory microRNA (e.g., (miR)-21). Repeated treatments with RigantmiR-21 significantly increased type I IFN production and cytotoxicity of cancer cells and improved the survival of tumor-bearing mice compared with repeated treatments with conventional RIG-I agonists.

The inventors have surprisingly discovered that RNA molecules that contain both PRR-activating motifs (e.g., RIG-I-activating motif(s)) and a complementary sequence to mature microRNA (e.g., miR-21 or mature miR146a) have superior anti-tumor and innate immune stimulatory effects to conventional PRR agonists. The inventors also discovered that innate immune cells and cancer cells with prior activation of PRRs or tryptophan metabolism signaling undergo PRR tolerance and diminish their ability to produce type I IFNs.

### Compositions

The present disclosure provides in part a nucleic acid molecule comprising a pattern recognition receptor (PRR) agonist and a microRNA antagonist.

The term “nucleic acid” or “nucleic acid molecule” refers to any of deoxyribonucleic acid (DNA), ribonucleic acid (RNA), oligonucleotides, fragments generated, for example, by chemical synthesis, polymerase chain reaction (PCR) or by in vitro transcription, and fragments generated by any one or more of ligation, scission, endonuclease action, or exonuclease action. Nucleic acid molecules can be composed of monomers that are naturally occurring nucleotides (such as deoxyribonucleotides and ribonucleotides), analogs of naturally occurring nucleotides (e.g., α-enantiomeric forms of naturally-occurring nucleotides), or a combination thereof. Modified nucleotides can have modifications of hydroxy groups in sugar moieties, or pyrimidine or purine base moieties with, e.g., halo (e.g. fluoro), amino, or thiol groups or combinations thereof. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, morpholino, or the like. Nucleic acid molecules can be either single stranded or double stranded. Additionally, nucleic acid molecules can be RNA, DNA, an aptamer, an oligonucleotide or an unmethylated polynucleotide molecule. The nucleic acid molecule can also comprises a 5′ triphosphate (5′ ppp).

In some embodiments, the nucleic acid molecule is polyinosinic-polycytidylic acid (polyI:C), pIC-HMW (high molecular weight), pIC-LMW (low molecular weight), 2′3′-cGAMP or c-di-GMP.

The term “nucleotide” refers to sequences with conventional nucleotide bases, sugar residues and internucleotide phosphate linkages, but also to those that contain modifications of any or all of these moieties. The term “nucleotide” as used herein includes those moieties that contain not only the natively found purine and pyrimidine bases adenine (A), guanine (G), thymine (T), cytosine (C), and uracil (U), but also modified or analogous forms thereof. Polynucleotides include RNA and DNA sequences of more than one nucleotide in a single chain. Modified RNA or modified DNA, as used herein, refers to a nucleic acid molecule in which one or more of the components of the nucleic acid, namely sugars, bases, and phosphate moieties, are different from that which occurs in nature.

A “complementary sequence” refers to a nucleic acid sequence that can form a double-stranded structure by matching base pairs. For example, the complementary sequence to C-A-T-G (where each letter stands for one of the bases in DNA) is G-T-A-C. As another example, the complementary sequence to C-A-U-G (where each letter stands for one of the bases in RNA) is G-U-A-C.

In some embodiments, the nucleic acid molecule can be nuclease resistant. Nucleases (e.g., DNase or RNase) are enzymes that degrade nucleic acids, and thus, a nuclease resistant nucleic acid molecule is capable of avoiding being degraded by nucleases.

The term “pattern recognition receptor (PRR)” refers to proteins that play a role in the proper function of the innate immune system. PRRs can identify damage-associated molecular patterns (DAMPs), which are associated with components of cells that are released during cell damage or death and can initiate and perpetuate a noninfectious inflammatory response. PRRs can also identify pathogen-associated molecular patterns (PAMPs), molecules present in viruses and bacteria. Examples of a PRR include, but are not limited to, retinoic acid-inducible gene I (RIG-I), Stimulator of Interferon Genes (STING), Melanoma Differentiation Associated Protein -5 (MDA5), Laboratory of Genetics and Physiology 2 (LPG2), toll-like receptors (TLR) (e.g., TLR1, TLR2, TLR3, TLR4, TLR5, TLR6, TLR7, TLR8, TLR9, TLR10, TLR11, and TLR13), C-type lectin receptors (CLR), RNA-activated protein kinase R (PKR), nucleotide-binding oligomerization domain-containing protein 2 (NOD2), Nacht leucine-rich repeat protein 3 (NALP3), interferon-induced protein with tetratricopeptide repeats 1 (IFIT1) and combinations thereof. In some embodiments, the PRR is RIG-I.

In some embodiments, the PPR comprises a cytoplasmic RNA-sensing PRR. The term “cytoplasmic RNA-sensing PRR” refers to a PRR capable of detecting various RNA structures in the cytoplasm. Examples of cytoplasmic RNA-sensing PRRs include, but are not limited to, RIG-I, MDA5, LPG2, PKR, NOD2, NALP3, and IFIT1.

RIG-I is an RNA-sensing PRR that can bind to ligands that include, but are not limited to, short double stranded RNA molecules (e.g., about 23-30 base pairs in length), 5′ di-phosphate (5′-pp) or tri-phosphate (5′-ppp) RNA, RNase L cleavage product, or circular RNA.

MDA5 is an RNA-sensing PRR that can bind to ligands that include, but are not limited to, long double stranded RNA (e.g., greater than 200 base pairs in length).

The term “agonist” refers to a substance that can initiate a physiological response when combined with a receptor. For example, a PRR agonist can be a nucleic acid molecule, protein, polysaccharide, or small molecule that is capable of activating PRRs that trigger different innate immune signaling pathways. In some embodiments, a PRR agonist can be immunogenic cell-killing RNA. Examples of various immunogenic cell-killing RNA sequences and structural features of these RNAs are set forth in International Patent Application Publication No. WO 2018/187328 and U.S. Patent Publication No. US 2018-0200183 A1, which are both incorporated herein by reference in their entireties.

Examples of immunogenic cell-killing RNA RIG-I agonists include, but are not limited to, the nucleic acid molecules sequences set forth in SEQ ID NO: 1, (referred to herein as ICR4), SEQ ID NO: 5 (referred to herein as ICR2), SEQ ID NO: 10 (referred to herein as ICR2AS), or any variants, portions, mutants, or fragments thereof. The sequences are provided in Table 1.

TABLE 1
RIG-I Agonist Sequences

| PRR | |
|---|---|
| Agonist | Sequence |
| ICR4 | 5′ GGAUGCGGUACCUGA |
| | CAGCAUCCUAAACUCAUG |
| | GUCCAUGUUUGUCCAUGG |
| | ACCA-3′ |
| | (SEQ ID: 1) |
| ICR2 | 5′-GGAUGCGGUACCUGA |
| | CAGCAUCC-3′ |
| | (SEQ ID NO: 5) |
| ICR2AS | 5′-ppp-GGAUGCUGUCA |
| | GGUACCGCAUCC-3′ |
| | (SEQ ID NO: 10) |

In some embodiments, the nucleic acid molecule comprises a PRR agonists having a nucleic acid sequence set forth in SEQ ID NO: 1 (referred to herein as ICR4), SEQ ID NO: 5 (referred to herein as ICR2), or SEQ ID NO: 10 (referred to herein as ICR2AS), or having at least 50%, 60%, 70%, 80%, 85%, 86%, 87%, 88%, 89%, 90%, 91%, 92%, 93%, 94%, 95%, 96%, 97%, 98%, or 99% sequence identity to the nucleic acid sequence set forth in SEQ ID NO:1, SEQ ID NO:5, or SEQ ID NO:10, or any variants, portions, mutants, or fragments thereof.

The term “sequence identity” refers to the number of identical or similar nucleotide bases on a comparison between a test and reference oligonucleotide or nucleotide sequence. Sequence identity can be determined by sequence alignment of nucleic acid to identify regions of similarity or identity. As described herein, sequence identity is generally determined by alignment to identify identical residues. Matches, mismatches, and gaps can be identified between compared sequences. Alternatively, sequence identity can be determined without taking into account gaps as the number of identical positions/length of the total aligned sequence×100. In one non-limiting embodiment, the term “at least 90% sequence identity to” refers to percent identities from 90 to 100%, relative to the reference nucleotide sequence. Identity at a level of 90% or more is indicative of the fact that, assuming for exemplary purposes a test and reference oligonucleotide length of 100 nucleotides are compared, no more than 10% (i.e., 10 out of 100) of the nucleotides in the test oligonucleotide differ from those of the reference oligonucleotide. Differences are defined as nucleic acid substitutions, insertions, or deletions.

Examples of MDA5 agonists include, but are not limited to, polyinosinic-polycytidylic acid (polyI:C), pIC-HMW (high molecular weight; 1500-8000 base pairs), and pIC-LMW (low molecular weight; 200-1000 base pairs).

Examples of a STING agonist include, but are not limited to, 2′3′-cGAMP, 3′3′-cGAMP, 2′2′ -cGAMP, -cAIMP, c-di-GMP, and c-di-AMP.

Examples of a TLR4 agonist include, but are not limited to, HMGB1, monophosphoryl lipid A, heparan sulfate, and LPS.

Examples of TLR9 agonists include CpG oligodeoxynucleotides.

As used herein, the term “microRNA antagonist” refers to a complementary sequence to a mature microRNA.

As used herein, the term “miRNA” or “miR” or “microRNA” or “mature RNA” refers to a non-coding RNA between 10 and 30 nucleotides in length which hybridizes to and regulates the expression of a coding RNA (see, Zeng and Cullen, RNA, 9(1): 112-123, 2003; Kidner and Martienssen Trends Genet, 19(1): 13-6, 2003; Dennis C, Nature, 420(6917):732, 2002; Couzin J, Science 298(5602):2296-7, 2002, each of which is incorporated by reference herein). A 10 to 30 nucleotide miRNA molecule can be obtained from a miRNA precursor through natural processing means (e.g., using intact cells or cell lysates) or by synthetic processing routes (e.g., using isolated processing enzymes, such as isolated Dicer, Argonaut, or RNAse III). It is understood that the 10 to 30 nucleotide RNA molecule can also be produced directly or by biological or chemical synthesis, without having been processed from a miR precursor. Included within this definition is natural miRNA molecules, pre-miRNA, pri-miRNA, miRNA molecules identical in nucleic acid sequence to the natural forms as well as nucleic acid sequences, where in one or more nucleic acids has been replaced or is represented by one or more DNA nucleotides and/or nucleic acid analogue. miRNA molecules in the present specification are occasionally referred to as a nucleic acid molecule(s) encoding a miRNA or simply nucleic acid molecule(s). Suitable examples of miRNA include, but are not limited to, those miRNAs found at www.mirbase.org/cgi-bin/mirna_summary.pl?org=hsa.

Exemplary miRNAs include, but are not limited to miR-21 and miR-146a.

In some embodiments, the microRNA antagonist is a complementary sequence to a mature microRNA selected from the group consisting of miR-21, miR-146a, and combinations thereof. In other embodiments, the microRNA antagonist is a complementary sequence to mature miR-21. In yet other embodiments, the microRNA antagonist is a complementary sequence to mature miR-146a.

The term “antagonist” refers to a substance that reduces, inhibits, or blocks the effects of a receptor. For example, a microRNA antagonist can be a complementary sequence to a mature microRNA (e.g., miR-21) that reduces, inhibits, or blocks the mature microRNA (e.g., reduces, inhibits, or blocks the immune regulatory and/or oncogenic function of miR-21).

In some embodiments, the microRNA antagonist comprises the nucleic acid sequence 5′-UCAACAUCAGUCUGAUAAGCUA-3′ (SEQ ID NO:11), the nucleic acid sequence 5′-ACAGCCCAUCGACUGGUGUUG-3′ (SEQ ID NO:12) or a nucleic acid sequence having at least 50%, 60%, 70%, 80%, 85%, 86%, 87%, 88%, 89%, 90%, 91%, 92%, 93%, 94%, 95%, 96%, 97%, 98%, or 99% sequence identity to the nucleic acid sequence set forth in SEQ ID NO:11 or SEQ ID NO:12, or any variants, portions, mutants, or fragments thereof.

In some embodiments, the microRNA antagonist comprises the sequence 5′-AACCCAUGGAAUUCAGUUCUCA-3′ (SEQ ID NO:13), the nucleic acid sequence 5′-CUGAAGAACUGAAUUUCAGAGG-3′ (SEQ ID NO:14) or a nucleic acid sequence having at least 50%, 60%, 70%, 80%, 85%, 86%, 87%, 88%, 89%, 90%, 91%, 92%, 93%, 94%, 95%, 96%, 97%, 98%, or 99% sequence identity to the nucleic acid sequence set forth in SEQ ID NO: 13 or SEQ ID NO:14, or any variants, portions, mutants, or fragments thereof.

In some embodiments, the nucleic acid molecule comprises a RIG-I agonist nucleic acid sequence and a miR-21 antagonist nucleic acid sequence. Examples of a nucleic acid molecule comprising a RIG-I agonist sequence and a miR-21 antagonist sequence include, but are not limited to, SEQ ID NO: 2 (referred to herein as RigantmiR-21), SEQ ID NO: 3 (referred to herein as mutant RigantmiR-21), SEQ ID NO: 6 (referred to herein as the ICR2-mutant anti-miR-21 sequence), SEQ ID NO: 7 (referred to herein as ICR2-anti-miR-21 sequence), SEQ ID NO: 8 (referred to herein as the ICR2AS-mutant anti-miR-21 sequence), SEQ ID NO: 9 (referred to herein as the ICR2AS-anti-miR-21 sequence), or or any variants, portions, mutants, or fragments thereof. The sequences for exemplary nucleic acid molecules comprising a RIG-I agonist sequence and a miR-21 antagonist sequence are set forth in Table 2.

TABLE 2
RIG-I agonist/miR-21
antagonist sequences

| Sequence | |
|---|---|
| name | Sequence |
| RigantmiR-21 | 5′- |
| | GGAUGCGGUACCUG |
| | ACAGCAUCUUGAAA |
| | UAAGGACUGAUGCU |
| | CAACAUCAGUCUGA |
| | UAAGCUA-3′ |
| | (SEQ ID NO: 2) |
| Mutant | 5′- |
| RigantmiR-21 | GGAUGCGGUACCUG |
| | ACAGCAUCUUGAAA |
| | UAAGGACUGAUGCU |
| | CAACAUCAGUCUGA |
| | CACUCCA-3′ |
| | (SEQ ID NO: 3) |
| ICR2-mutant | 5′- |
| Anti-miR-21 | GGAUGCGGUACCUG |
| | ACAGCAUCCUCAAC |
| | AUCAGUCUGCAGCCG |
| | AG-3′ |
| | (SEQ ID NO: 6) |
| ICR2-anti- | 5′- |
| miR-21 | GGAUGCGGUACCUGA |
| | CAGCAUCCUCAACAU |
| | CAGUCUGAUAAGC |
| | UA-3′ |
| | (SEQ ID NO: 7), |
| ICR2AS- | 5′- |
| mutant-anti- | GGAUGCUGUCAGGUA |
| miR-21 | CCGCAUCCUCAACAU |
| | CAGUCUGCAGCCG |
| | AG-3′ |
| | (SEQ ID NO: 8) |
| ICR2AS-anti- | 5′- |
| miR-21 | GGAUGCUGUCAGGUA |
| | CCGCAUCCUCAACAU |
| | CAGUCUGAUAAGC |
| | UA-3′ |
| | (SEQ ID NO: 9) |

In some embodiments, the nucleic acid molecule comprises the sequence set forth in SEQ ID NO:2, SEQ ID NO:3, SEQ ID NO:6, SEQ ID NO:7, SEQ ID NO: 8, SEQ ID NO: 9, or a nucleic acid sequence having at least 50%, 60%, 70%, 80%, 85%, 86%, 87%, 88%, 89%, 90%, 91%, 92%, 93%, 94%, 95%, 96%, 97%, 98%, or 99% sequence identity to the nucleic acid sequence set forth in SEQ ID NO:2, SEQ ID NO:3, SEQ ID NO:6, SEQ ID NO:7, SEQ ID NO: 8, SEQ ID NO: 9, or any variants, portions, mutants, or fragments thereof.

In other embodiments, the nucleic acid molecule comprises a RIG-I agonist sequence and a miR-146a antagonist sequence. Examples of a nucleic acid molecule comprising a RIG-I agonist sequence and a miR-146a antagonist sequence include, but are not limited to, the sequence set forth in SEQ ID NO: 4 (referred to herein as RigantimiR-146a). The sequences for exemplary nucleic acid molecules comprising a RIG-I agonist sequence and a miR-146a antagonist sequence are set forth in Table 3.

| Sequence | |
|---|---|
| name | Sequence |
| RigantimiR- | 5′- |
| 146a | GGAUGCGGUACCUG |
| | ACAGCAUCCUCCCC |
| | CGCAUUCCAUGGUC |
| | AACCCAUGGAAUUC |
| | AGUUCUCA-3′ |
| | (SEQ ID NO: 4) |

In some embodiments, the nucleic acid molecule comprises the sequence set forth in SEQ ID NO:4, or a nucleic acid sequence having at least 50%, 60%, 70%, 80%, 85%, 86%, 87%, 88%, 89%, 90%, 91%, 92%, 93%, 94%, 95%, 96%, 97%, 98%, or 99% sequence identity to the nucleic acid sequence set forth in SEQ ID NO: 4, or any variants, portions, mutants, or fragments thereof.

In some embodiments, the PRR agonist is not a nucleic acid molecule. For example, the PRR agonist can be a protein, lipopolysaccharide (LPS), or small molecule. Examples of non-nucleic acid PRR agonists include, but are not limited to high mobility group box 1 (HMGB1) and LPS. When the PRR agonist is a non-nucleic acid molecule, this molecule can be “conjugated” to a microRNA antagonist to form a “PRR agonist/microRNA antagonist conjugate.” It will be appreciated that a protein, lipopolysaccharide, or small molecule PRR agonist can be conjugated to a microRNA antagonist using techniques known in the art.

In some embodiments, the PRR agonist is a protein (e.g., HMGB1), lipopolysaccharide (e.g., LPS, monophosphoryl lipid A, or heparan sulfate), or small molecule that is conjugated to a microRNA antagonist (e.g., a complementary sequence to miR-21 and miR-146a).

Another aspect of the present disclosure provides a composition comprising a nucleic acid molecule comprising a pattern recognition receptor (PRR) agonist and a microRNA antagonist. Yet another aspect of the present disclosure provides a composition comprising a PRR agonist/microRNA antagonist conjugate.

In some embodiments, the compositions can further comprise a pharmaceutically acceptable carrier and/or excipient.

A “pharmaceutically acceptable excipient and/or carrier” includes, but is not limited to, sterile distilled water, saline, phosphate buffered solutions, amino acid-based buffers, or bicarbonate buffered solutions. Other suitable excipient and/or carriers may include any solvent, dispersion medium, vehicle, coating, diluent, antibacterial, and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the active ingredient, its use in the therapeutic compositions is contemplated. Supplementary active ingredients also can be incorporated into the compositions. An excipient and/or carrier selected and the amount of excipient and/or used will depend upon the mode of administration. Administration comprises an injection, infusion, or a combination thereof.

The compositions described herein can also include a cytoplasmic delivery mechanism. Such delivery mechanisms are available to those skilled in the art and include all gene delivery mechanisms including but not limited to synthetic polymers (e.g., those used for siRNA delivery), cell-penetrating peptides (e.g., VP16), nanoparticles, viral or liposomal delivery to the cytoplasm of cells (e.g., lipofection), delivery via a gene gun, or may include transfection, nucleofection or electroporation. The cytoplasmic delivery mechanisms may be targeted to only deliver the compositions to cells in which cell growth inhibition or induction of programmed cell death is desired. For example, the cellular delivery mechanism may specifically target the RNAs to cancer cells. The compositions may be targeted to cells for uptake by receptor-mediated endocytosis as well. In addition, cells could be genetically engineered to express the RNA compositions described herein. The RNAs could be operably connected to a promoter, such as an inducible promoter, to allow expression of the RNA only upon proper stimulation.

The compositions can also comprise a cytoplasmic delivery composition. A cytoplasmic delivery composition can deliver exogenous nucleic acids such as DNA, RNA or oligonucleotides into cells. The cytoplasmic delivery composition can be a liposome, a synthetic polymer, a cell-penetrating peptide, a nanoparticle, a viral particle, an electroporation buffer, a nucleofection reagent, or any combination thereof. In some embodiments, a cytoplasmic delivery composition is referred to as a transfection agent. Examples of cytoplasmic delivery compositions include, but are not limited to DharmaFECT and jet-PEI.

In some embodiments, the composition is a pharmaceutical composition comprising the nucleic acid molecule and a pharmaceutically acceptable carrier and/or excipient.

### Methods

The nucleic acid molecules, PRR agonist/microRNA antagonist conjugate molecules, compositions/pharmaceutical compositions of the present disclosure may further be used in various methods.

In one aspect, the present disclosure provides a method of treating or preventing cancer in a subject in need thereof, the method comprising, consisting of, or consisting essentially of administering to the subject a therapeutically effective amount of a nucleic acid molecule comprising a pattern recognition receptor (PRR) agonist and a microRNA antagonist or a PRR agonist/microRNA antagonist conjugate molecule such that the cancer is treated or prevented in the subject.

As used herein, “treatment,” “treating,” “therapy,” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

The molecules and compositions provided herein can be administered to a subject, either alone or in combination with a pharmaceutically acceptable excipient/carrier, in an amount sufficient to induce an appropriate anti-tumor response. The response can comprise, without limitation, specific immune response, non-specific immune response, both specific and non-specific response, innate response, primary immune response, adaptive immunity, secondary immune response, memory immune response, immune cell activation, immune cell proliferation, immune cell differentiation, and cytokine expression.

Accordingly, the present disclosure provides methods of providing an anti-tumor immunity in a subject by administering to the subject an effective amount of the nucleic acid molecules and/or compositions and/or pharmaceutical compositions as provided herein. An “effective amount” or “therapeutically effective amount” as used herein means an amount which provides a therapeutic or prophylactic benefit. Effective amounts of the nucleic acid molecules and/or compositions and/or pharmaceutical compositions can be determined by a physician with consideration of individual differences in age, weight, tumor size, extent of infection or metastasis, and condition of the patient (subject).

It can generally be stated that a pharmaceutical composition comprising the compositions/pharmaceutical compositions described herein may be in a concentration of 1 ng/mL to 100 ng/mL, 1 μg/mL to 100 μg/ML, 1 mg/mL to 130 mg/mL, 10 mg/mL to 130 mg/mL, 40 mg/mL to 120 mg/mL, 80 mg/mL to 110 mg/mL, about 1 mg/mL to about 130 mg/mL, about 10 mg/mL to about 130 mg/mL, about 40 mg/mL to about 120 mg/mL, or about 80 mg/mL to about 110 mg/mL. In some embodiments, the compound is present in a concentration of 10 mg/mL, 20 mg/mL, 30 mg/mL, 40 mg/mL, 50 mg/mL, 60 mg/mL, 70 mg/mL, 80 mg/mL, 90 mg/mL, 100 mg/mL, 110 mg/mL, 120 mg/mL, 130 mg/mL, 140 mg/mL, 150 mg/mL, 160 mg/mL, 170 mg/mL, 180 mg/mL, 190 mg/mL, 200 mg/mL, about 10 mg/mL, about 20 mg/mL, about 30 mg/mL, about 40 mg/mL, about 50 mg/mL, about 60 mg/mL, about 70 mg/mL, about 80 mg/mL, about 90 mg/mL, about 100 mg/mL, about 110 mg/mL, about 120 mg/mL, about 130 mg/mL, about 140 mg/mL, about 150 mg/mL, about 160 mg/mL, about 170 mg/mL, about 180 mg/mL, about 190 mg/mL, or about 200 mg/mL. In some embodiments, the compound is present in a concentration of 100 mg/mL. In some embodiments, the compound is present in a concentration of about 100 mg/mL.

The optimal dosage and treatment regime for a particular patient can readily be determined by one skilled in the art of medicine by monitoring the patient for signs of disease and adjusting the treatment accordingly. In certain embodiments, the nucleic acid molecules and/or compositions of the invention may be administered at a dose of 1 mg/kg body weight to 100 mg/kg, 1 mg/kg to 50 mg/kg, 1 mg/kg to 25 mg/kg, or 1 mg/kg to 10 mg/kg.

An effective amount of the nucleic acid molecules and/or compositions described herein may be given in one dose, but is not restricted to one dose. Thus, the administration can be two, three, four, five, six, seven, eight, nine, ten, eleven, twelve, thirteen, fourteen, fifteen, sixteen, seventeen, eighteen, nineteen, twenty, or more, administrations of the composition. Where there is more than one administration in the present methods, the administrations can be spaced by time intervals of one minute, two minutes, three, four, five, six, seven, eight, nine, ten, or more minutes, by intervals of about one hour, two hours, three, four, five, six, seven, eight, nine, ten, 11, 12, 13, 14, 15, 16, 17, 18, 19, 20, 21, 22, 23, 24 hours, and so on. In the context of hours, the term “about” means plus or minus any time interval within 30 minutes. The administrations can also be spaced by time intervals of one day, two days, three days, four days, five days, six days, seven days, eight days, nine days, ten days, 11 days, 12 days, 13 days, 14 days, 15 days, 16 days, 17 days, 18 days, 19 days, 20 days, 21 days, and combinations thereof. The present disclosure is not limited to dosing intervals that are spaced equally in time, but encompass doses at non-equal intervals, such as a priming schedule consisting of administration at 1 day, 4 days, 7 days, and 25 days, just to provide a non-limiting example.

An effective amount of a therapeutic agent is one that will decrease or ameliorate the symptoms normally by at least 10%, more normally by at least 20%, most normally by at least 30%, typically by at least 40%, more typically by at least 50%, most typically by at least 60%, often by at least 70%, more often by at least 80%, and most often by at least 90%, conventionally by at least 95%, more conventionally by at least 99%, and most conventionally by at least 99.9%.

The term “disease” as used herein includes, but is not limited to, any abnormal condition and/or disorder of a structure or a function that affects a part of an organism. It may be caused by an external factor, such as an infectious disease, or by internal dysfunctions, such as cancer, cancer metastasis, and the like.

As is known in the art, a cancer is generally considered as uncontrolled cell growth. The nucleic acid molecules, compositions, and methods of the present disclosure can be used to treat any cancer, and any metastases thereof, including, but not limited to, carcinoma, lymphoma, blastoma, sarcoma, and leukemia. More particular examples of such cancers include, but are not limited to, breast cancer, prostate cancer, colon cancer, squamous cell cancer, small-cell lung cancer, non-small cell lung cancer, ovarian cancer, cervical cancer, gastrointestinal cancer, pancreatic cancer, glioblastoma, liver cancer, bladder cancer, hepatoma, colorectal cancer, uterine cervical cancer, endometrial carcinoma, salivary gland carcinoma, mesothelioma, kidney cancer, vulval cancer, pancreatic cancer, thyroid cancer, hepatic carcinoma, skin cancer, melanoma, brain cancer, neuroblastoma, glioblastoma, myeloma, various types of head and neck cancer, acute lymphoblastic leukemia, acute myeloid leukemia, lymphoma, soft tissue sarcoma, osteosarcoma, Ewing sarcoma, and peripheral neuroepithelioma. In some embodiments, the cancer comprises a type of cancer that are resistant to checkpoint inhibitor immunotherapy, RIG-1 therapy, MDA5 therapy, PRR therapy, and/or STING therapy.

As used herein, the term “cancer cell” refers to one or more cells of cancer, and can include one or more cells from any of the exemplary cancers described herein. The term cancer cell can also refer to cancer cell lines. Examples of cancer cell lines include, but are not limited to human cancer cell lines (e.g., melanoma cell lines: WM226.4, WM115, SK-MEL2, MALME; pancreatic cell lines: Panc-1; cervical cancer cell lines: Hela; breast cancer cell lines Hs578T; sarcoma cell lines: HT1080; ovarian cancer cell lines: SK-OV-3; lung cancer cell lines: NCI-H1838; prostate cancer cell lines: DU145, PC3, LNCaP; glioma cell lines: D392; glioblsatoma cell lines: LN229, Xeno43, and liver cancer cell lines: Huh7) and murine cancer cell lines (e.g., melanoma cancer cell lines: B16-F0, B16-F10; sarcoma cell lines: 403688, 403754, 402230; H&N cell lines: 40616, 40828; breast cancer cell lines: 4T1; and pancreatic cancer cell lines: PANC-02).

It will be appreciated that normal cell lines (e.g., normal human cell lines) can be used as a control to determine the effects of any of the disclosed nucleic acid molecules, PRR agonist/microRNA antagonist conjugate molecules, or compositions/pharmaceutical compositions on a cancer cell line. Examples of normal human cell lines include, but are not limited to, melanocyte, dermal fibroblast, lung fibroblast, colon epithelia, prostate epithelia, bone marrow stroma, and PBMC cell lines.

An effective amount for a particular subject/patient may vary depending on factors such as the condition being treated, the overall health of the patient, the route and dose of administration and the severity of side effects. Guidance for methods of treatment and diagnosis is available (see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK).

A dosing schedule of, for example, once/week, twice/week, three times/week, four times/week, five times/week, six times/week, seven times/week, once every two weeks, once every three weeks, once every four weeks, once every five weeks, and the like, is available for the invention. The dosing schedules encompass dosing for a total period of time of, for example, one week, two weeks, three weeks, four weeks, five weeks, six weeks, two months, three months, four months, five months, six months, seven months, eight months, nine months, ten months, eleven months, and twelve months.

Provided are cycles of the above dosing schedules. The cycle can be repeated about, e.g., every seven days; every 14 days; every 21 days; every 28 days; every 35 days; 42 days; every 49 days; every 56 days; every 63 days; every 70 days; and the like. An interval of non-dosing can occur between a cycle, where the interval can be about, e.g., seven days; 14 days; 21 days; 28 days; 35 days; 42 days; 49 days; 56 days; 63 days; 70 days; and the like. In this context, the term “about” means plus or minus one day, plus or minus two days, plus or minus three days, plus or minus four days, plus or minus five days, plus or minus six days, or plus or minus seven days.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. In some embodiments, the subject is a human. In one embodiment, the subject is a human suffering from a cancer. In another embodiment, the subject is a human suffering from a type of cancer that is resistant to checkpoint inhibitor immunotherapy, RIG-1 therapy, MDA5 therapy, PRR therapy, and/or STING therapy.

The term “administration” or “administering” as it applies to a human, primate, mammal, mammalian subject, animal, veterinary subject, placebo subject, research subject, experimental subject, cell, tissue, organ, or biological fluid, refers without limitation to contact of an exogenous ligand, reagent, placebo, small molecule, pharmaceutical agent, therapeutic agent, diagnostic agent, or composition to the subject, cell, tissue, organ, or biological fluid, and the like. “Administration” can refer, e.g., to therapeutic, pharmacokinetic, diagnostic, research, placebo, and experimental methods. Treatment of a cell encompasses exposure of the cell to a reagent (e.g., a nucleic acid molecule), as well as contact of a reagent to a fluid, where the fluid is in contact with the cell. “Administering” also encompasses in vitro and ex vivo treatments, e.g., of a cell, by a reagent, diagnostic, binding composition, or by another cell.

Modes of administration include oral, rectal, transmucosal, topical, transdermal, inhalation, parenteral, intravenous, subcutaneous, intradermal, intramuscular and intraarticular administration, and the like, as well as directly into tissue (e.g., muscle) or organ injection (e.g., into an organ containing cancer cells), intrathecal, intraventricular, intraperitoneal, intranasal, intraocular, or intratumoral.

Thus, the compositions can be formulated as an ingestible, injectable, topical, or suppository formulation. These formulations can be prepared in conventional forms, either as liquid solutions or suspensions, solid forms suitable for solution or suspension in liquid prior to administration, or as emulsions. An injection medium will typically be an aqueous liquid that contains the additives usual for injection solutions, such as stabilizing agents, salts or saline, and/or buffers.

In another aspect, the present disclosure provides a method of treating cancers resistant to immune checkpoint blockade therapy, RIG-1-therapy, MDA5 therapy, and/or STING therapy in a subject, the method comprising, consisting of, or consisting essentially of administering to the subject a therapeutically effective amount of a nucleic acid molecule comprising a pattern recognition receptor (PRR) agonist and a microRNA antagonist or a PRR agonist/microRNA antagonist conjugate molecule such that the cancer is prevented in the subject.

In another aspect, the present disclosure provides a method of sensitizing a cancer cell to a PRR agonist, the method comprising, consisting of, or consisting essentially of, exposing the cancer cell to a nucleic acid molecule comprising a pattern recognition receptor (PRR) agonist and a microRNA antagonist or a PRR agonist/microRNA antagonist conjugate molecule.

The term “sensitizing” refers to causing a cancer cell to have an improved response to exposure to a PRR agonist.

Another aspect of the disclosure provides a method of sensitizing a cancer cell to a PRR agonist, the method comprising, consisting of, or consisting essentially of, exposing the cancer cell to a nucleic acid molecule comprising a pattern recognition receptor (PRR) agonist and a microRNA antagonist or a PRR agonist/microRNA antagonist conjugate molecule.

Yet another aspect of the disclosure provides a method of improving an anticancer innate immune response to a cancer cell, the method comprising, consisting of, or consisting essentially of, exposing the cancer cell to a nucleic acid molecule comprising a pattern recognition receptor (PRR) agonist and a microRNA antagonist or a PRR agonist/microRNA antagonist conjugate molecule. The term “improving” in this context refers to increasing the anticancer innate immune response to a cancer cell. Improving can also mean that the anticancer innate immune response to a cancer cell is stimulated.

Another aspect of the disclosure provides a method of mitigating miR-21 or miR-146a interference with the antitumor effects of a RIG-I agonist in a cancer cell, the method comprising, consisting of, or consisting essentially of exposing the cancer cell to a nucleic acid molecule comprising a pattern recognition receptor (PRR) agonist and a microRNA antagonist or a PRR agonist/microRNA antagonist conjugate molecule.

Yet another aspect of the disclosure provides a method of mitigating miR-21 or miR-146a interference with the antitumor effects of a RIG-I agonist in a cancer cell, the method comprising, consisting of, or consisting essentially of exposing the cancer cell to a nucleic acid molecule comprising a pattern recognition receptor (PRR) agonist and a microRNA antagonist or a PRR agonist/microRNA antagonist conjugate molecule.

In some embodiments, the nucleic acid molecules, compositions according to the present disclosure may also be administered with one or more additional therapeutic agents (e.g., tryptophan signaling inhibitors, IDO inhibitor, TDO inhibitor, AHR inhibitor, other chemotherapeutic agents, adjuvants, immune checkpoint inhibitors (e.g., anti-PD-1 antibody, anti-PDL-1 inhibitors, and anti-CTLA4 inhibitors), and the like). Methods for co-administration with an additional therapeutic agents are well known in the art (Hardman, et al. (eds.) (2001) Goodman and Gilman's The Pharmacological Basis of Therapeutics, 10th ed., McGraw-Hill, New York, N.Y.; Poole and Peterson (eds.) (2001) Pharmacotherapeutics for Advanced Practice:A Practical Approach, Lippincott, Williams & Wilkins, Phila., Pa.; Chabner and Longo (eds.) (2001) Cancer Chemotherapy and Biotherapy, Lippincott, Williams & Wilkins, Phila., Pa.).

Co-administration need not refer to administration at the same time in an individual, but rather may include administrations that are spaced by hours or even days, weeks, or longer, as long as the administration of multiple therapeutic agents is the result of a single treatment plan. The co-administration may comprise administering the compositions of the present disclosure before, after, or at the same time as the one or more additional therapeutic agents. In one example of a treatment schedule, the compositions of the present disclosure may be given as an initial dose in a multi-day protocol, with one or more additional therapeutic agents given on later administration days; or the one or more additional therapeutic agents given as an initial dose in a multi-day protocol, with the compositions of the present disclosure given on later administration days. On another hand, one or more additional therapeutic agents and the compositions(s) of the present disclosure may be administered on alternate days in a multi-day protocol. In still another example, a mixture of one or more additional therapeutic agents and the compositions of the present disclosure may be administered to the subject. This is not meant to be a limiting list of possible administration protocols.

In some embodiments, the subject is pretreated with one or more tryptophan signaling inhibitors. In one embodiment, the tryptophan signaling inhibitor comprises inhibitors of indoleamine 2,3-dioxygenase (IDO). Any suitable IDO inhibitor may be used according to the present disclosure. Also suitable are inhibitors of IDO isoenzymes, including for example tryptophan (2,3)-dioxygenase (TDO) IDO1, and/or IDO2. Thus, the IDO inhibitor for use with the combinations of the invention may inhibit, directly or indirectly, IDO1 and/or TDO and/or IDO2. Suitable IDO inhibitors include those based on natural products, such as the cabbage extract brassinin, the marine hydroid extract annulin B and the marine sponge extract exiguamine A, including synthetic derivatives thereof. Other suitable IDO inhibitors include molecular analogues of its substrate, tryptophan. Such inhibitors include the tryptophan mimetic 1-methyl tryptophan (1-MT). 1-MT occurs as two stereoisomers: the L isomer significantly inhibits IDO1, while the D isomer is more specific for IDO2. The D isomer (D-1-MT, indoximod) is currently being evaluated in a phase II, double-blind, randomized, placebo-controlled trial. Other suitable IDO inhibitors include INCB24360, a hydroxyamidine small-molecule inhibitor, and GDC-0919. Unlike 1-MT-based inhibitors, hydroxyamidine inhibitors also inhibit tryptophan (2,3)-dioxygenase (TDO), an enzyme with identical activity to IDO. Yet another suitable IDO inhibitor is NLG919. In other embodiments, the IDO inhibitor comprises 1-methyltryptophan (1-MT).

In other embodiments, the tryptophan signaling inhibitor comprises an AHR inhibitor. In certain embodiments, the AHR inhibitor comprises CH223191 or BAY-218.

Formulations of one or more additional therapeutic agents and/or the compositions as provided herein may be prepared for storage by mixing with physiologically acceptable carriers, excipients, or stabilizers in the form of, e.g., lyophilized powders, slurries, aqueous solutions or suspensions (see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.).

### Kits

Other aspects of the present disclosure provides a kit for the prevention and/or treatment of a cancer in subject, the kit comprising, consisting of, or consisting essentially of a composition as provided herein and instructions for use.

Yet another aspect of the present disclosure provides all that is disclosed and illustrated herein.

The following Examples are provided by way of illustration and not by way of limitation.

### EXAMPLES

### Example 1

### RigantmiR-21 Can Overcome Resistance of Cancer Cells to RIG-I-Mediated Anticancer Therapy

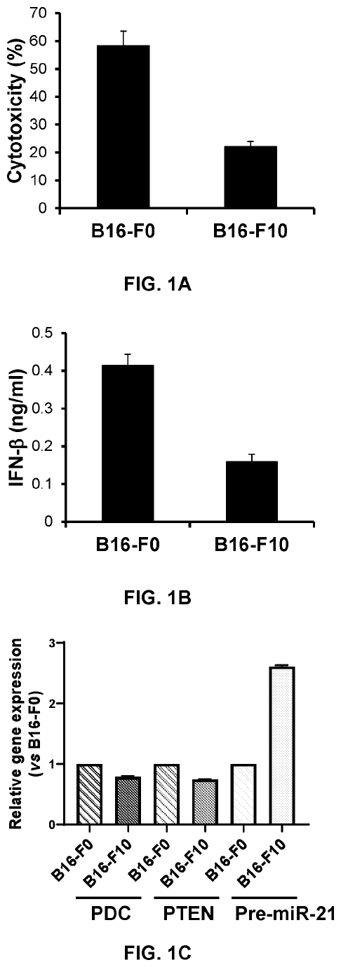

B16-F0 and B16-F10 mouse melanoma cells lines are originated from the same parental cell line but they have different metastatic ability. B16-F0 cell line has no or marginal metastatic ability but B16-F10 cell line has potent metastatic ability. B16-F0 and B16-F10 cells (1×10⁴) were treated with RIG-I agonist ICR4-liposome complex (0.6 μg/ml). After overnight incubation, cytotoxicity and IFN-β production by the cells were measured using MTT assay and ELISA, respectively. Although both B16-F0 and B16-F10 cell lines underwent cell death and IFN-β production upon treatment with the RIG-I agonist ICR4⁴, the B16-F0 cell line was much more sensitive to antitumor effects of ICR4 than the B16-F10 cell line as shown in FIGS. 1A and 1B. Tumor suppressor proteins, such as programmed cell death 4 (PDCD4) and phosphatase and tensin homolog (PTEN), and pre-miR-21 levels in B16-F0 and B16-F10 cells were measured using real-time qPCR. Interestingly, as shown in FIG. 1C, B16-F10 cells express 2-3 fold higher level of premiR-21 than B16-F0 cells. Moreover, miR-21 target genes, such as programmed cell death 4 (PDCD4) and phosphatase and tensin homolog (PTEN), were downregulated in B16-F10 cells compared to B16-F0 cells. It was hypothisized that miR-21 would interfere with antitumor effects of RIG-I agonists in B16-F10 melanoma cells.

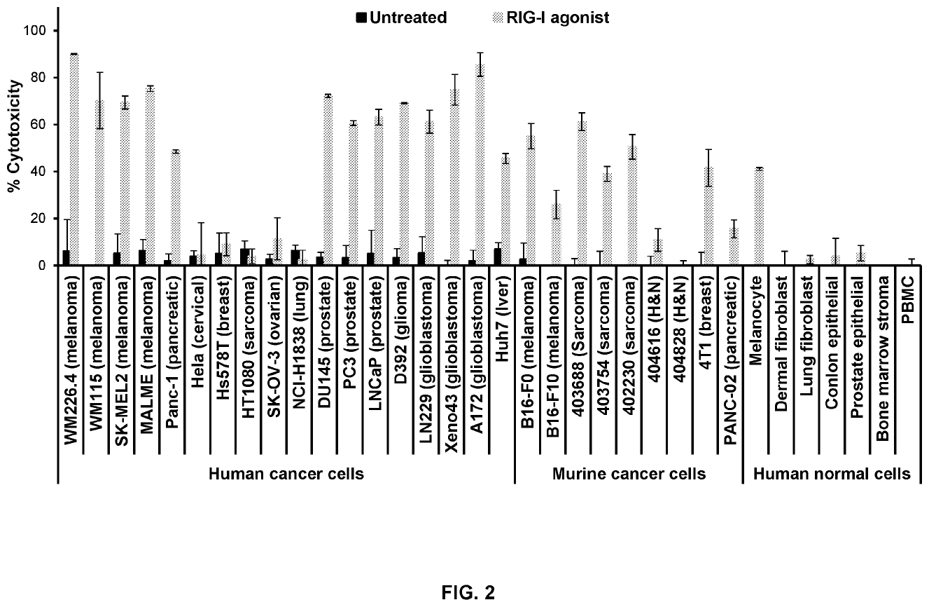

Additionally, cancer and normal human cells were determined to be differentially sensitive to cytotoxicity of RIG-I agonists. Human and murine cancer cell lines and human normal cells in 96-well plate (7000 cells/well) were transfected with RIG-I agonist 5′ppp ssRNA ICR4 (0.8 mg/ml). Cells were harvested 72 h after treatment and analyzed for cytotoxicity using MTT assay. N=3. *, P<0.05. Results (% cytotoxicity) are shown in FIG. 2.

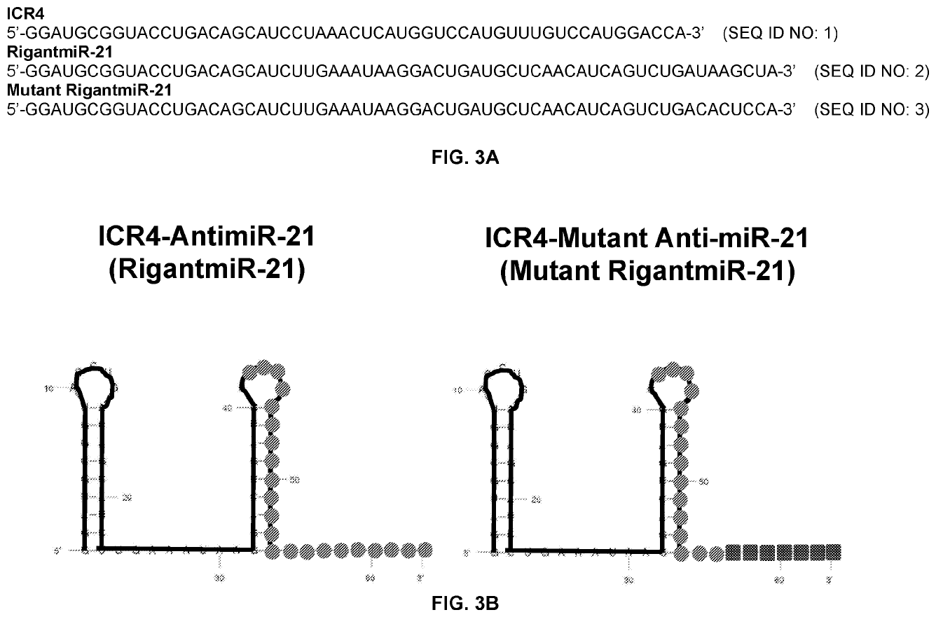

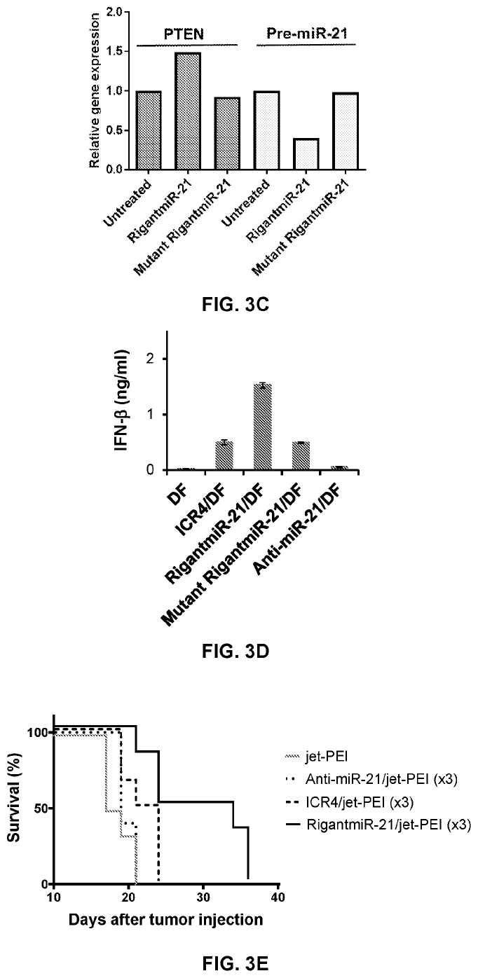

To improve therapeutic effects of RIG-I agonists in B16-F10 cells, we designed and generated a novel RIG-I-activating RNA molecule (named RigantmiR-21) that is composed of a RIG-I-activating RNA structure (ICR4: 5′ppp-containg double stem-loop RNA) and a complementary sequence to mature miR-21. See FIGS. 3A and 3B. B16-F10 melanoma cells were transfected once or twice with RigantmiR-21, mutant RigantmiR-21, anti-miR-21, or ICR4 (0.6 μg/ml) using liposomal transfection agent DharmaFECT (DF). The expression of miR-21 target genes was determined using qPCR. IFN-β production was determined using ELISA. Interestingly, RigantmiR-21 treatment inhibited the expression of premiR-21 and upregulated miR-21 target gene (PTEN) expression in B16-F10 melanoma cells as shown in FIG. 3C. This RigantmiR-21 treatment significantly increased IFN-β production by B16-F10 cells compared to ICR4 and mutant RigantmiR-21 that is composed of ICR4 and a non-complementary sequence to miR-21 as shown in FIG. 3D. Anti-miR-21, ICR4, and RigantmiR-21 RNAs were complexed with in vivo transfection agent jet-PEI. Jet-PEI alone or RNA-jet-PEI complexes (25 μg) were injected thrice intratumorally into B16-F10 melanoma-bearing immunocompetent mice. (n=6). Repeated intratumoral injections with RigantmiR-21 significantly improved the survival of B16-F10 melanoma-bearing mice compared to repeated injections with ICR4 or anti-miR-21 as shown in FIG. 3E.

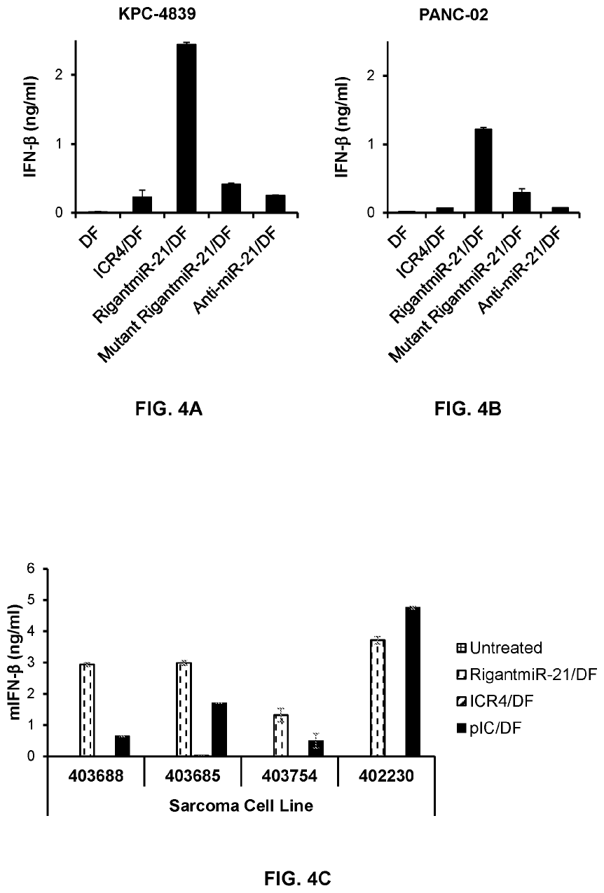

The antitumor effects of RigantmiR-21 are not limited to melanoma cells. KPC-4839 (mouse pancreatic ductal adenocarcinoma), PANC-02 (mouse pancreatic adenocarcinoma) and mouse sarcoma cell line cells were transfected with indicated ICR4, RigantmiR-21, mutant RigantmiR-21, or anti-miR-21 (0.6 μg/ml) using DharmaFECT (DF). After overnight incubation, IFN-β production by these cells was determined using ELSIA. (n=3). As shown in FIGS. 4A-4C, RigantmiR-21 treatments induced cell death and IFN-β production by mouse pancreatic, adenocarcinoma, and sarcoma cells that are also resistant to RIG-I agonist ICR4. The antitumor activities of RigantmiR-21 were comparable to those of MDA5 agonist polyI:C.

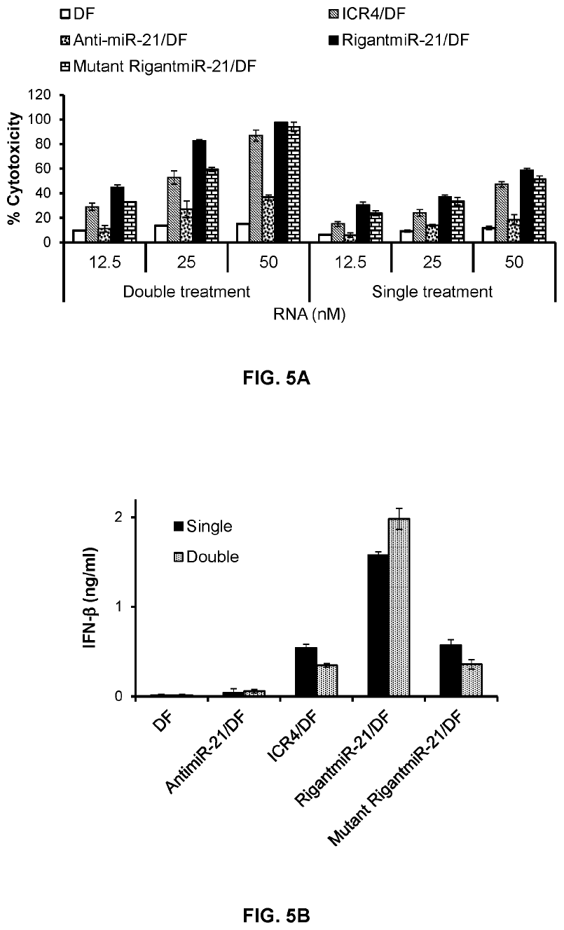

Interestingly, cytotoxic effects of RigantmiR-21, mutant RigantmiR-21, anti-miR-21, and ICR4 were gradually augmented in cancer cells after repeated treatments, as shown in FIG. 5A. B16-F0 mouse melanoma cells were transfected with ICR4, RigantmiR-21, mutant RigantmiR-21, or anti-miR-21 at various concentrations using DharmaFECT (DF). After overnight incubation, the cells were replenished with fresh culture media, followed by another transfection with the same dose of RNAs. Three days after the first transfection, cytotoxicity levels were measured using MTT assay.

By contrast, anti-miR-21 did not induce IFN-β production by cancer cells, as shown in FIG. 5B. Furthermore, melanoma cells pre-treated with ICR4 or mutant RigantmiR-21 showed significantly reduced IFN-β production while these cells pre-treated with RigantmiR-21 showed incremental IFN-β production. B16-F0 mouse melanoma cells were transfected with ICR4, RigantmiR-21, mutant RigantmiR-21, or anti-miR-21 at 12.5 nM using DharmaFECT (DF). After overnight incubation, the cells were replenished with fresh culture media, followed by another transfection with the same dose of RNAs. One day after the first and second transfection, IFN-β production by these cells were determined using ELSIA.

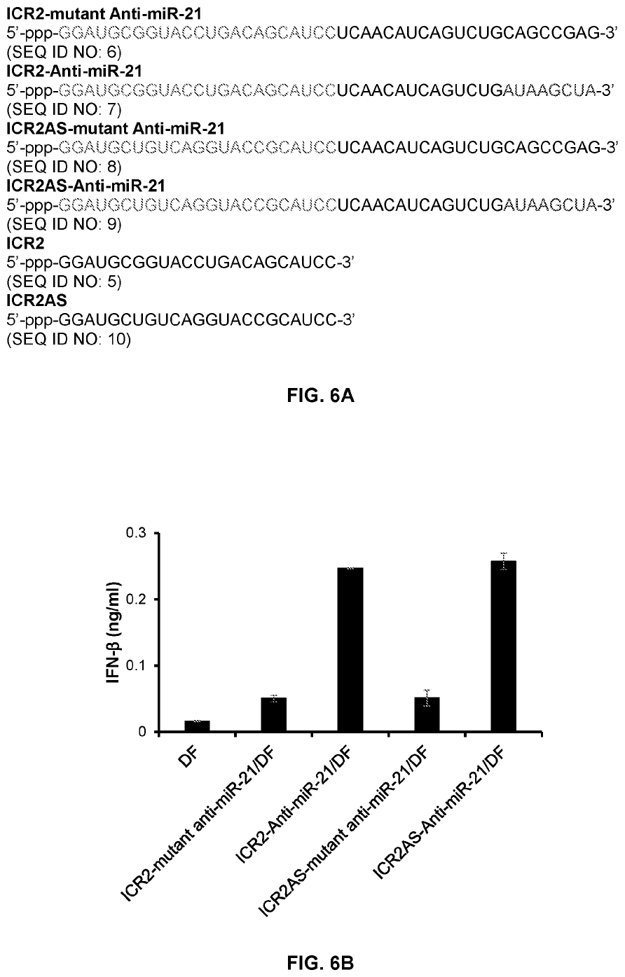

Additionally, conjugation of anti-miR-21 improved the ability of other types of RIG-I agonists, such as ICR2 and ICR2AS⁴ (sequences shown in FIG. 6A) to induce IFN-β production by cancer cells. FIG. 6B shows the increased induction of IFN-β production by B16-F10 mouse melanoma cells compared to ICR2 and ICR2As conjugated with seed mutant anti-miR-21 (n=3).

### Example 2

### Prior Activation of Innate Immune Receptors and/or Tryptophan Metabolism Signaling Pathways Induces Tolerance of Cytoplasmic Nucleic Acid-Sensing RIG-I, MDA5, and STING in Cancer Cells and Immune Cells

It has been shown that treatments with RNA-sensing innate immune receptor agonists induce a short period of increased innate immune stimulation followed by tolerance lasting several days, leading to reduced immunostimulatory cytokine production by dendritic cells (DCs), decreased tumor-specific CD8+ T cells, and diminished inhibition of tumor growth in tumor-bearing mice^{58,59}.

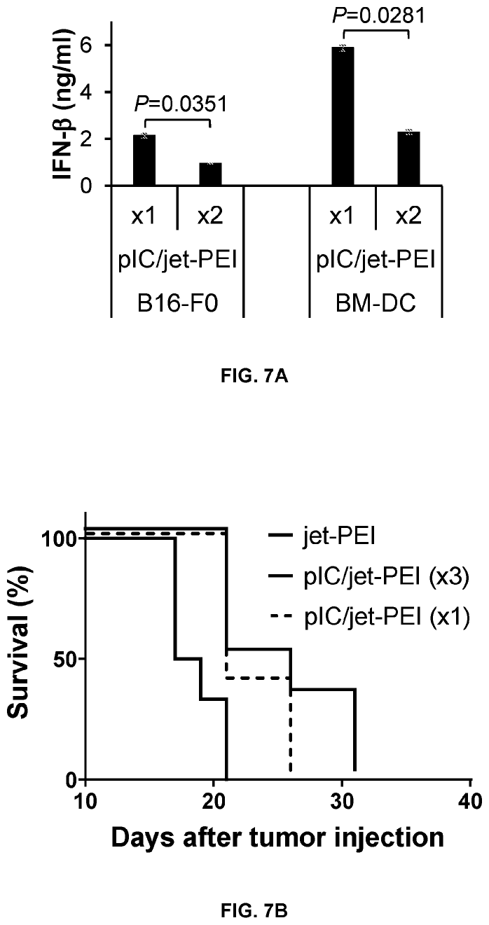

B16-F0 mouse melanoma cells or bone marrow-derived dendritic cells (BM-DCs) were transfected once (x1) or twice (x2) with a complex of polyI:C (pIC) (0.6 μg/ml) and transfection agent jet-PEI. IFN-β production by the cells one day after each transfection was determined using ELISA. Consistent with previous studies, and as shown in FIG. 7A, prior activation of RNA-sensing MDA5 induced a state of hyporesponsiveness in mouse melanoma cells and DCs, leading to reduced IFN-β production by these cells after retreatment with MDA5 agonists. Thereby, multiple treatments with MDA5 agonists did not significantly enhance anticancer responses in B16-F10 melanoma-bearing mice compared to single treatment with MDA5 agonists as shown in FIG. 7B, where pIC/jet-PEI complex (25 μg) was injected once (x1 ) or thrice (x3) intratumorally into B16-F0 melanoma-bearing mice (n=6).

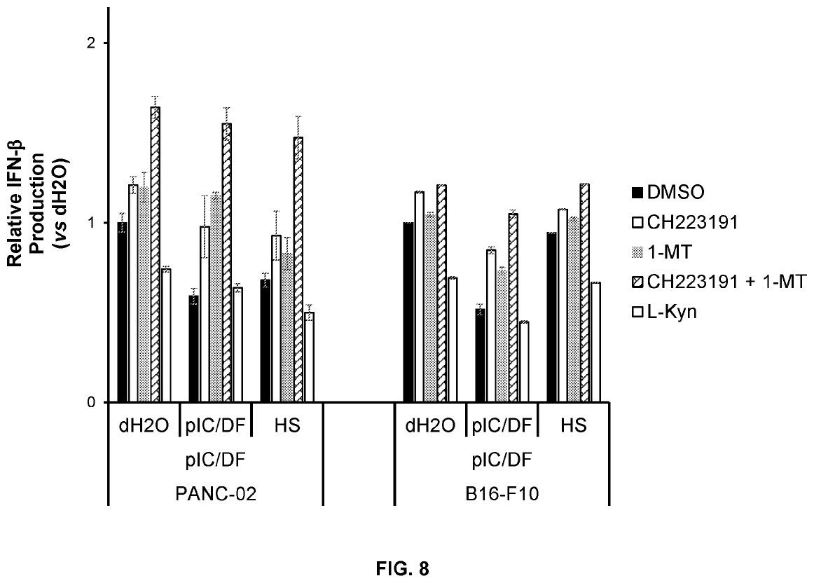

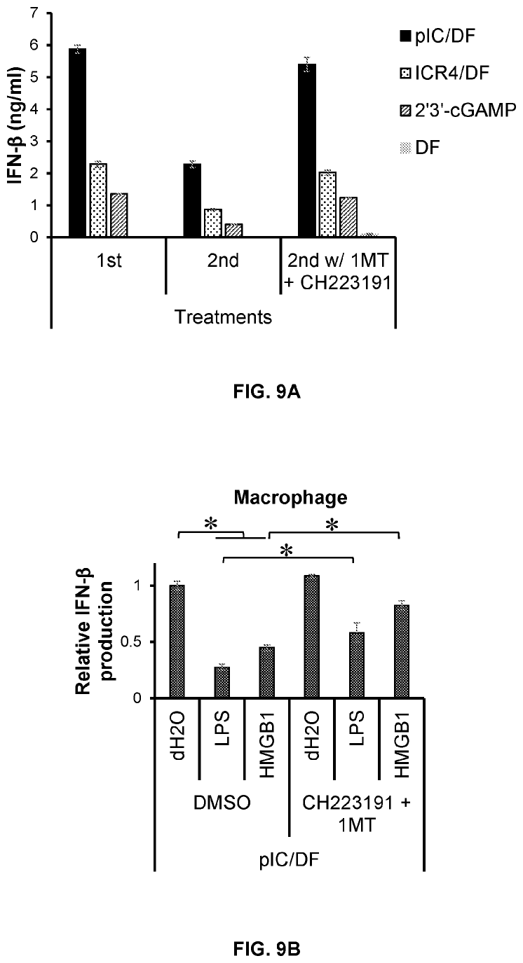

In addition to prior activation of MDA5, prior activation of TLR4, RIG-I, and STING induced self- and cross-tolerance of same and different PRRs. Mouse cancer cell lines, such as PANC-02 and B16-F10, and innate immune cells, such as DC and macrophage, pretreated with MDA5 agonist (pIC/DF), RIG-I agonist (ICR4/DF), STING agonist (2′3′-cGAMP), or TLR4 agonists (HMGB1 and LPS) at a sublethal dose produced significantly decreased IFN-β after secondary treatment with MDA5, RIG-I, or STING agonist compared to the cells pretreated with the control dH₂O as shown in FIG. 8 and FIG. 9.

In particular, mouse pancreatic cancer cell line PANC-02 and melanoma cell line B16-F10 were pretreated with dH₂O, MDA5 agonist (pIC/DF complex), or TLR4 agonist (HMGB1) in the presence of dimethyl sulfoxide (DMSO) (vehicle control), CH223191 (AHR inhibitor), 1-MT (IDO inhibitor), or L-Kyn (AHR activator). After overnight incubation, the cells were replenished with fresh culture media and treated with pIC/DF complex (0.6 μg/ml). One day after second treatment, IFN-β production was measured using ELISA. (n=3). These results are shown in FIG. 8.

Mouse BM-DCs were treated with pIC/DF complex (cytoplasmic MDA5), ICR4/DF complex (cytoplasmic RIG-I), 2′3′-cGAMP (cytoplasmic STING), or transfection agent DF alone (FIG. 9A). Mouse macrophage cells were treated with dH₂O (control), LPS (surface TLR4), or HMGB1 (surface TLR4), followed by treatment with DMSO (control) or mixture of CH223191 (AHR inhibitor) and 1-MT (IDO inhibitor) (FIG. 9B). After overnight incubation, the cells were replenished with fresh culture media and treated with indicated cytoplasmic PRR agonists. Levels of IFN-β production were measured using ELISA. □ P<0.05.

Interestingly, naive cancer cells pre-treated with tryptophan catabolite Kyn also showed significantly decreased IFN-β production after PRR agonist treatment (FIG. 8). By contrast, cancer cells and innate immune cells pre-treated with a mixture of tryptophan signaling inhibitors, such as IDO inhibitor (1-methyltrypophan (1-MT)) and AHR inhibitor (CH223191), showed significantly increased IFN-β production compared to the cells pre-treated with control dimethyl sulphoxide (DMSO) (FIG. 8 and FIG. 9).

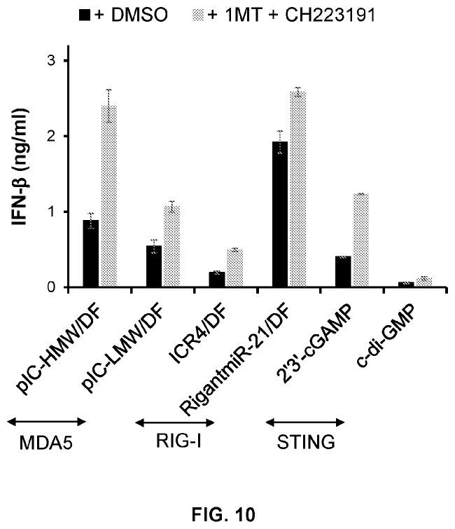

Furthermore, co-treatments of IDO inhibitor, AHR inhibitor, and cytoplasmic PRR agonists significantly augmented IFN-β production by innate immune cells compared to PRR agonists alone (FIG. 10). Finally, IDO inhibitor and AHR inhibitor co-treatment enhanced innate immune stimulatory effects of RigantmiR-21 (FIG. 10). Mouse macrophage cells were treated with MDA5 agonists (pIC-HMW and pIC-LMW), RIG-I agonists (ICR4 and RigantmiR-21), and STING agonists (2′3′-cGAMP and c-di-GMP). After overnight incubation, the cells were replenished with fresh culture media and treated with the same agonists in the presence or absence of a mixture of 1-MT and CH223191. (n=3). These results are shown in FIG. 10.

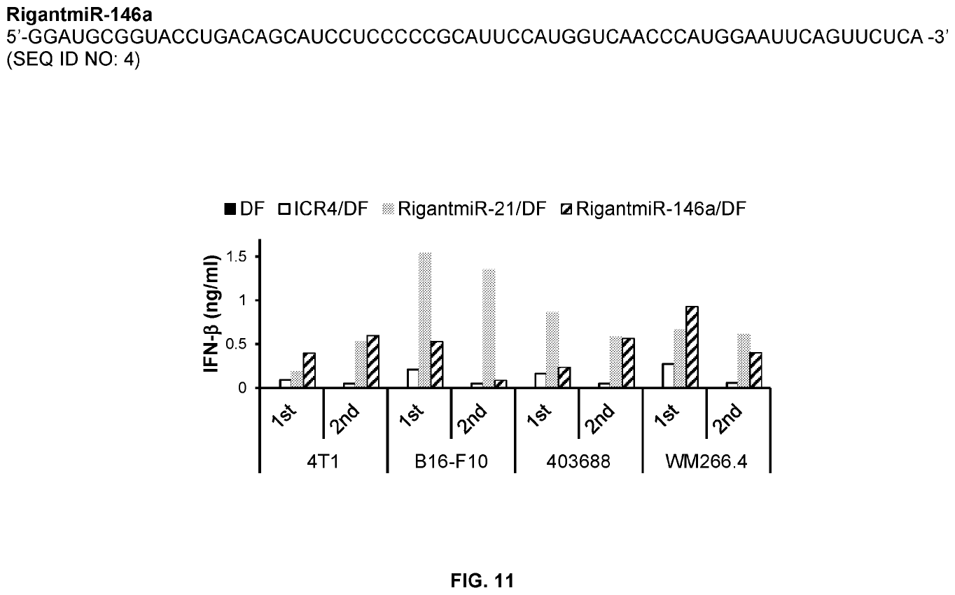

Additionally, it was demonstrated that RignatmiR-21 and RigantmiR-146a differentially induce IFN-β production by different cancer cells. 4T1 mouse breast cancer cells, B16-F10 mouse melanoma cells, 403688 mouse sarcoma cells, and WM266.4 human melanoma cells were transfected with indicated RIG-I agonists using liposomal transfection agent DharmaFECT (DF). After overnight incubation, the cells was replenished with fresh culture media, followed by another transfection with the same RIG-I agonists. One day after first and second transfection IFN-β production by these cells were determined using ELSIA. Results are shown in FIG. 11.

### Example 3

### RigantmiR-21 Synergizes with Anti-PD-1 Antibody for the Treatment of Mouse Sarcoma

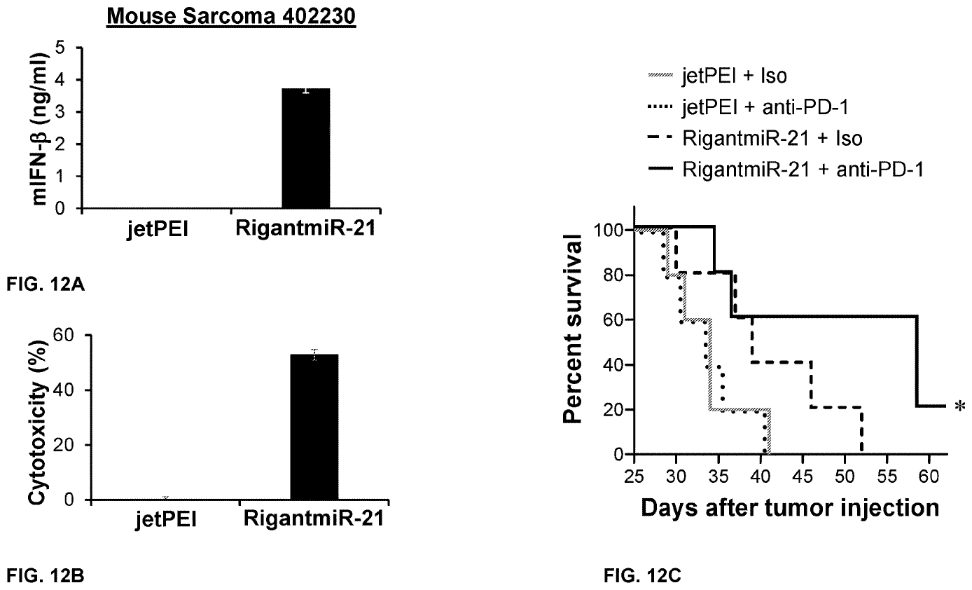

Surprisingly, RigantmiR-21 synergizes with anti-PD-1 antibody for the treatment of mouse sarcoma. Murine sarcoma 402230 cell line was treated single with RigantmiR-21 (0.8 mg/ml) in complex with liposome. IFN-β production 24 h after treatment (FIG. 12A) and cytotoxicity 72 h after treatment (FIG. 12B) were measured using ELISA and MTT assay, respectively. N=3. 402230 sarcoma-bearing immunocompetent mice were injected intratumorally once daily for three days with RigantmiR-21 (1 mg/kg) in complex with in vivo transfection agent jetPEI, N=5. (FIG. 12C). Sarcoma-bearing mice were also injected intraperitoneally twice at 7-day interval with anti-PD-1. jetPEI alone and jetPEI with isotype control antibody were used as control. Injections with RigantmiR-21 and anti-PD-1 antibody significantly improved survival of 402230 sarcoma-bearing immunocompetent mice compared to RigantmiR-21 alone.

### REFERENCES

1. Duewell, P., et al. Targeted activation of melanoma differentiation-associated protein 5 (MDA5) for immunotherapy of pancreatic carcinoma. Oncoimmunology 4, e1029698 (2015).

2. Duewell, P., et al. RIG-I-like helicases induce immunogenic cell death of pancreatic cancer cells and sensitize tumors toward killing by CD8(+) T cells. Cell Death Differ 21, 1825-1837 (2014).

3. Poeck, H., et al. 5′-Triphosphate-siRNA: turning gene silencing and Rig-I activation against melanoma. Nat Med 14, 1256-1263 (2008).

4. Lee, J., Lee, Y., Xu, L., White, R. & Sullenger, B. A. Differential Induction of Immunogenic Cell Death and Interferon Expression in Cancer Cells by Structured ssRNAs. Mol Ther 25, 1295-1305 (2017).

5. Apetoh, L., et al. Toll-like receptor 4-dependent contribution of the immune system to anticancer chemotherapy and radiotherapy. Nat Med 13, 1050-1059 (2007).

6. Dunn, G. P., et al. A critical function for type I interferons in cancer immunoediting. Nat Immunol 6, 722-729 (2005).

7. Burnette, B. C., et al. The efficacy of radiotherapy relies upon induction of type i interferon-dependent innate and adaptive immunity. Cancer Res 71, 2488-2496 (2011).

8. Diamond, M. S., et al. Type I interferon is selectively required by dendritic cells for immune rejection of tumors. J Exp Med 208, 1989-2003 (2011).

9. Fuertes, M. B., et al. Host type I IFN signals are required for antitumor CD8+ T cell responses through CD8{alpha}+ dendritic cells. J Exp Med 208, 2005-2016 (2011).

10. Deng, L., et al. STING-Dependent Cytosolic DNA Sensing Promotes Radiation-Induced Type I Interferon-Dependent Antitumor Immunity in Immunogenic Tumors. Immunity 41, 843-852 (2014).

11. Krysko, D. V., et al. Immunogenic cell death and DAMPs in cancer therapy. Nat Rev Cancer 12, 860-875 (2012).

12. Elliott, M. R., et al. Nucleotides released by apoptotic cells act as a find-me signal to promote phagocytic clearance. Nature 461, 282-286 (2009).

13. Gardai, S. J., et al. Cell-surface calreticulin initiates clearance of viable or apoptotic cells through trans-activation of LRP on the phagocyte. Cell 123, 321-334 (2005).

14. Sistigu, A., et al. Cancer cell-autonomous contribution of type I interferon signaling to the efficacy of chemotherapy. Nat Med 20, 1301-1309 (2014).

15. Musella, M., Manic, G., De Maria, R., Vitale, I. & Sistigu, A. Type-I-interferons in infection and cancer: Unanticipated dynamics with therapeutic implications. Oncoimmunology 6, e1314424 (2017).

16. Kroemer, G., Galluzzi, L., Kepp, O. & Zitvogel, L. Immunogenic cell death in cancer therapy. Annu Rev Immunol 31, 51-72 (2013).

17. Deng, L., et al. Irradiation and anti-PD-L1 treatment synergistically promote antitumor immunity in mice. J Clin Invest 124, 687-695 (2014).

18. Gajewski, T. F. The Next Hurdle in Cancer Immunotherapy: Overcoming the Non-T-Cell-Inflamed Tumor Microenvironment. Semin Oncol 42, 663-671 (2015).

19. Bald, T., et al. Immune cell-poor melanomas benefit from PD-1 blockade after targeted type I IFN activation. Cancer Discov 4, 674-687 (2014).

20. Kawai, T. & Akira, S. TLR signaling. Semin Immunol 19, 24-32 (2007).

21. Venereau, E., Ceriotti, C. & Bianchi, M. E. DAMPs from Cell Death to New Life. Front Immunol 6, 422 (2015).

22. Lotze, M. T., et al. The grateful dead: damage-associated molecular pattern molecules and reduction/oxidation regulate immunity. Immunological reviews 220, 60-81 (2007).

23. Pichlmair, A., et al. RIG-I-mediated antiviral responses to single-stranded RNA bearing 5′-phosphates. Science 314, 997-1001 (2006).

24. Hornung, V., et al. 5′-Triphosphate RNA is the ligand for RIG-I. Science 314, 994-997 (2006).

25. Kato, H., et al. Length-dependent recognition of double-stranded ribonucleic acids by retinoic acid-inducible gene-I and melanoma differentiation-associated gene 5. The Journal of experimental medicine 205, 1601-1610 (2008).

26. Tanaka, Y. & Chen, Z. J. STING specifies IRF3 phosphorylation by TBK1 in the cytosolic DNA signaling pathway. Sci Signal 5, ra20 (2012).

27. Leulier, F. & Lemaitre, B. Toll-like receptors—taking an evolutionary approach. Nat Rev Genet 9, 165-178 (2008).

28. Feldman, N., Rotter-Maskowitz, A. & Okun, E. DAMPs as mediators of sterile inflammation in aging-related pathologies. Ageing research reviews 24, 29-39 (2015).

29. Yamamoto, M., et al. Cutting edge: a novel Toll/IL-1 receptor domain-containing adapter that preferentially activates the IFN-beta promoter in the Toll-like receptor signaling. J Immunol 169, 6668-6672 (2002).

30. Yamamoto, M., et al. Role of adaptor TRIF in the MyD88-independent toll-like receptor signaling pathway. Science 301, 640-643 (2003).

31. Oshiumi, H., Matsumoto, M., Funami, K., Akazawa, T. & Seya, T. TICAM-1, an adaptor molecule that participates in Toll-like receptor 3-mediated interferon-beta induction. Nat Immunol 4, 161-167 (2003).

32. O'Neill, L. A. & Bowie, A. G. The family of five: TIR-domain-containing adaptors in Toll-like receptor signalling. Nat Rev Immunol 7, 353-364 (2007).

33. Cao, X. Self-regulation and cross-regulation of pattern-recognition receptor signalling in health and disease. Nat Rev Immunol 16, 35-50 (2016).

34. Kobayashi, K., et al. IRAK-M is a negative regulator of Toll-like receptor signaling. Cell 110, 191-202 (2002).

35. Xiong, Y., et al. Endotoxin tolerance impairs IL-1 receptor-associated kinase (IRAK) 4 and TGF-beta-activated kinase 1 activation, K63-linked polyubiquitination and assembly of IRAK1, TNF receptor-associated factor 6, and IkappaB kinase gamma and increases A20 expression. J Blot Chem 286, 7905-7916 (2011).

36. Murphy, M. B., et al. Pellino-3 promotes endotoxin tolerance and acts as a negative regulator of TLR2 and TLR4 signaling. J Leukoc Biol 98, 963-974 (2015).

37. Sheedy, F. J., et al. Negative regulation of TLR4 via targeting of the proinflammatory tumor suppressor PDCD4 by the microRNA miR-21. Nat Immunol 11, 141-147 (2010).

38. Nahid, M. A., Pauley, K. M., Satoh, M. & Chan, E. K. miR-146a is critical for endotoxin-induced tolerance: IMPLICATION IN INNATE IMMUNITY. J Biol Chem 284, 34590-34599 (2009).

39. de Vos, A. F., et al. In vivo lipopolysaccharide exposure of human blood leukocytes induces cross-tolerance to multiple TLR ligands. J Immunol 183, 533-542 (2009).

40. Puccetti, P. On watching the watchers: IDO and type I/II IFN. Eur J Immunol 37, 876-879 (2007).

41. Von Bubnoff, D., Scheler, M., Wilms, H., Fimmers, R. & Bieber, T. Identification of IDO-positive and IDO-negative human dendritic cells after activation by various proinflammatory stimuli. J Immunol 186, 6701-6709 (2011).

42. Huang, L., Xu, H. & Peng, G. TLR-mediated metabolic reprogramming in the tumor microenvironment: potential novel strategies for cancer immunotherapy. Cell Mol Immunol (2018).

43. Mellor, A. L., Keskin, D. B., Johnson, T., Chandler, P. & Munn, D. H. Cells expressing indoleamine 2,3-dioxygenase inhibit T cell responses. J Immunol 168, 3771-3776 (2002).

44. Quintana, F. J., et al. An endogenous aryl hydrocarbon receptor ligand acts on dendritic cells and T cells to suppress experimental autoimmune encephalomyelitis. Proc Natl Acad Sci USA 107, 20768-20773 (2010).

45. Uyttenhove, C., et al. Evidence for a tumoral immune resistance mechanism based on tryptophan degradation by indoleamine 2,3-dioxygenase. Nature Medicine 9, 1269 (2003).

46. Opitz, C. A., et al. An endogenous tumour-promoting ligand of the human aryl hydrocarbon receptor. Nature 478, 197-203 (2011).

47. Gandhi, R., et al. Activation of the aryl hydrocarbon receptor induces human type 1 regulatory T cell-like and Foxp3(+) regulatory T cells. Nat Immunol 11, 846-853 (2010).

48. Funatake, C. J., Marshall, N. B., Steppan, L. B., Mourich, D. V. & Kerkvliet, N. I. Cutting edge: activation of the aryl hydrocarbon receptor by 2,3,7,8-tetrachlorodibenzo-p-dioxin generates a population of CD4+ CD25+ cells with characteristics of regulatory T cells. J Immunol 175, 4184-4188 (2005).

49. Veldhoen, M., Hirota, K., Christensen, J., O'Garra, A. & Stockinger, B. Natural agonists for aryl hydrocarbon receptor in culture medium are essential for optimal differentiation of Th17 T cells. J Exp Med 206, 43-49 (2009).

50. Kimura, A., et al. Aryl hydrocarbon receptor in combination with Statl regulates LPS-induced inflammatory responses. J Exp Med 206, 2027-2035 (2009).

51. Bessede, A., et al. Aryl hydrocarbon receptor control of a disease tolerance defence pathway. Nature 511, 184-190 (2014).

52. Fallarino, F., et al. LPS-conditioned dendritic cells confer endotoxin tolerance contingent on tryptophan catabolism. Immunobiology 220, 315-321 (2015).

53. Yamada, T., et al. Constitutive aryl hydrocarbon receptor signaling constrains type I interferon-mediated antiviral innate defense. Nat Immunol 17, 687-694 (2016).

54. Platten, M., Wick, W. & Van den Eynde, B. J. Tryptophan catabolism in cancer: beyond IDO and tryptophan depletion. Cancer Res 72, 5435-5440 (2012).

55. Pfeffer, S. R., Yang, C. H. & Pfeffer, L. M. The Role of miR-21 in Cancer. Drug Dev Res 76, 270-277 (2015).

56. Mima, K., et al. MicroRNA MIR21 and T Cells in Colorectal Cancer. Cancer Immunol Res 4, 33-40 (2016).

57. Yang, C. H., Yue, J., Pfeffer, S. R., Handorf, C. R. & Pfeffer, L. M. MicroRNA miR-21 regulates the metastatic behavior of B16 melanoma cells. J Blot Chem 286, 39172-39178 (2011).

58. Bourquin, C., et al. Systemic cancer therapy with a small molecule agonist of toll-like receptor 7 can be improved by circumventing TLR tolerance. Cancer Res 71, 5123-5133 (2011).

59. Hotz, C., et al. Reprogramming of TLR7 signaling enhances antitumor NK and cytotoxic T cell responses. Oncoimmunology 5, e1232219 (2016).

1. A nucleic acid molecule comprising a pattern recognition receptor (PRR) agonist and a microRNA antagonist, wherein the PPR comprises a cytoplasmic RNA-sensing PRR.

2. (canceled)

3. The nucleic acid molecule of claim 1, wherein the PRR is selected from the group consisting of retinoic acid-inducible gene I (RIG-I), Stimulator of Interferon Genes (STING), Melanoma Differentiation Associated Protein-5 (MDA5), Laboratory of Genetics and Physiology 2 (LPG2), RNA-activated Protein Kinase (PKR), Nucleotide-binding Oligomerization Domain-containing Protein 2 (NOD2), Nacht Leucine-rich Protein 3 (NALP3), and combinations thereof.

4. The nucleic acid molecule of claim 1, wherein the PRR is RIG-I or MDA5.

5. The nucleic acid molecule of claim 1, wherein the microRNA antagonist is a complementary sequence to a mature microRNA selected from the group consisting of miR-21, miR-146a, and combinations thereof.

6.-11. (canceled)

12. The nucleic acid molecule of claim 1, wherein the nucleic acid molecule comprises a RIG-I agonist and a miR-21 antagonist.

13. The nucleic acid molecule of claim 1, wherein the nucleic acid molecule comprises a RIG-I agonist and a miR-146a antagonist.

14.-18. (canceled)

19. The nucleic acid molecule of claim 1, wherein the nucleic acid molecule comprises the sequence set forth in SEQ ID NO:2 or a sequence having at least 95% sequence identity to the sequence set forth in SEQ ID NO:2, or any variants, portions, mutants, or fragments thereof.

20. (canceled)

21. The nucleic acid molecule of claim 1, wherein the nucleic acid molecule comprises the sequence set forth in SEQ ID NO:4 or a sequence having at least 95% sequence identity to the sequence set forth in SEQ ID NO:4, or any variants, portions, mutants, or fragments thereof.

22.-36. (canceled)

37. A method of improving an anticancer innate immune response to a cancer cell, the method comprising exposing the cancer cell to a nucleic acid molecule comprising a pattern recognition receptor (PRR) agonist and a microRNA antagonist.

38.-39. (canceled)

40. The method of claim 37, wherein the cancer cell is a melanoma cancer cell, a pancreatic cancer cell, a cervival cancer cell, a breast cancer cell, a sarcoma cell, an ovarian cancer cell, a lung cancer cell, a prostate cancer cell, a glioma cancer cell, a glioblastoma cancer cell, or a liver cancer cell.

41. The method of claim 37, wherein the PPR comprises a cytoplasmic RNA-sensing PRR.

42. The method of claim 37, wherein the PRR is selected from the group consisting of retinoic acid-inducible gene I (RIG-I), Stimulator of Interferon Genes (STING), Melanoma Differentiation Associated Protein-5 (MDA5), Laboratory of Genetics and Physiology 2 (LPG2), and combinations thereof.

43. The method of claim 37, wherein the PRR is RIG-I or MDA5.

44. The method of claim 37, wherein the microRNA antagonist is a complementary sequence to a mature microRNA selected from the group consisting of miR-21, miR-146a, and combinations thereof.

45.-50. (canceled)

51. The method of claim 37, wherein the nucleic acid molecule comprises a RIG-I agonist and a miR-21 antagonist.

52. The method of claim 37, wherein the nucleic acid molecule comprises a RIG-I agonist and a miR-146a antagonist.

53.-57. (canceled)

58. The method of claim 37, wherein the nucleic acid molecule comprises the sequence set forth in SEQ ID NO:2 or a sequence having at least 95% sequence identity to the sequence set forth in SEQ ID NO:2, or any variants, portions, mutants, or fragments thereof.

59. (canceled)

60. The method of claim 37, wherein the nucleic acid molecule comprises the sequence set forth in SEQ ID NO:4 or a sequence having at least 95% sequence identity to the sequence set forth in SEQ ID NO:4, or any variants, portions, mutants, or fragments thereof.

61.-64. (canceled)

65. The method of claim 37, comprising contacting the cell with one or more tryptophan signaling inhibitors.

66. The method of claim 65, wherein the tryptophan signaling inhibitor comprises (i) an IDO inhibitor, wherein the IDO inhibitor comprises 1-methyltryptophan (1-MT), or (ii) an AHR inhibitor, wherein the AHR inhibitor comprises CH223191.

67.-71. (canceled)
